(19) United States
(12) Patent Application Publication
(10) Pub. No.: US 2023/0040211 A1
(43) Pub. Date: Feb. 9, 2023

(54) AUTOMATING VENTILATION USING AN AIRBAG

(71) Applicant: Darwood IP Limited, Colchester (GB)

(72) Inventor: Alastair Rupert Joseph DARWOOD, Colchester (GB)

(21) Appl. No.: 17/791,540

(22) PCT Filed: Jan. 11, 2021
(86) PCT No.: PCT/EP2021/050415

§ 371 Date: Jul. 8, 2022

(30) Foreign Application Priority Data
Jan. 10, 2020 (GB) 2000419.8

(51) Int. Cl. A61M 16/00

(52) U.S. Cl. CPC A61M 16/0084; A61M 16/022; A61M 2205/07; A61M 2016/0027; A61M 2205/106

(57) ABSTRACT

Apparatus is disclosed for ventilation using an airbag, which may also be operable manually. The apparatus comprises: means providing or penetrable to form an aperture in the airbag; retractable means provided separately from the airbag, for extending in the airbag; fixing means for fixing, by an operator, an end of the retractable means to the airbag via the aperture; actuation means, for repeatedly retracting the retractable means at least partially through the aperture in the airbag to collapse the airbag, and for enabling expansion of the airbag to an expanded state; and connecting means for connecting the actuation means to the airbag and for preventing environment egress of gas from within the airbag between the aperture and the actuation means.

### FIELD OF THE INVENTION

The invention relates to apparatus for automating ventilation using an airbag. In particular, the invention relates to automating repeated collapsing of an airbag of a manual ventilator such as a bag-valve-mask type ventilator.

### BACKGROUND

Manual resuscitation devices, such as bag-valve-mask (BVM) type ventilators, are ubiquitous in emergency management of patients requiring ventilatory support, such as apnoeic or bradypnoaeic patients. BVM's typically comprise a flexible, self-inflating airbag, a mask, a patient connection port connected to a mask, and an air input port. In use, the mask is sealed over a patient's face so that the bag can be squeezed to expel air within the bag through the patient connection port into the patient's lungs. The patient connection port is usually configured to allow gas to exit the airbag and enter a patient's lungs, and also to direct gas that emerges from a patient's lungs to the atmosphere. In use an attending clinician grasps the airbag and squeezes the airbag repeatedly in a controlled manner using one or two hands. Such compression action increases pressure within the airbag resulting in flow of gas from the airbag into the patients lungs, causing expansion of the lungs. When the compressive force on the airbag is reduced or removed, elastic re-expansion occurs drawing in fresh air via the air input port. The air input port may be coupled to an oxygen supply so that oxygen can be provided to the patient instead of or as a supplement to environmental air.

Whilst low cost and reliable, BVM ventilation is limited due to poor capability to control respiratory parameters, barotrauma, hypo/hyperventilation and also in that an attending clinician must attend solely to operation of the BVM as they are required to squeeze the bag and cannot attend to other clinically important matters. In view of this, patients are rapidly transferred and connected to dedicated pneumatic or digital portable ventilators, where available, capable of precise and safe control of ventilation for long periods of time. Whilst providing adequate ventilation, such ventilators are frequently high-cost, complex, fragile and bulky.

Devices for automating the ‘squeezing’ of a BVM device are described in the art, intended to provide a low cost alternative to conventional ventilators. For example, U.S. Pat. No. 8,534,282B2 describes a device into which an airbag may be inserted with apparatus to cyclically compress the airbag by means of a constraining strap and eccentric cam whereby the eccentric cam rotates and depresses a portion of the ventilator. This apparatus is necessarily bulky as external compression devices must be rigidly attached to the bag with suitable security whilst providing a stable foundation with which to compress the bag. Similarly, WO2016/203289AI describes a tabletop machine into which a bag may be placed capable of mechanically compressing the bag. These examples are poorly suited to the extreme portability and durability requirements of critical care ventilators.

U.S. Pat. No. 5,222,491A describes a ventilator having a guy wire based mechanism for compressing an airbag. The airbag has a hollow tube extending along a center axis of the airbag, from an air outlet to an airtight hole at the end of the airbag remote from the air outlet. Two guy wires extend radially from the hollow tube and attach at respectively opposing sides of the airbag. The guy wires are connected by a guy wire portion that extends through the hollow tube and through the airtight hole to a box, where pulling and release of the guy wire portion is controlled to, respectively, collapse and permit expansion of the resilient airbag. This ventilator is complex by comparison to other manual ventilators and is complicated by having the box collocated with one-way valve to enable air or oxygen to enter the airbag. Further, although the ventilator may be operated manually, the parts of the device that enable manual ventilation cannot be provided separately from additional parts that enable automated ventilation.

An object of the present invention is to address these issues.

### SUMMARY OF THE INVENTION

According to a first aspect of the present invention, there is provided apparatus for automating ventilation using an airbag, particularly for automating operation of an airbag of a manual ventilator, particularly for automating operation of an airbag of a manual ventilator, the apparatus comprising: means providing or penetrable to form an aperture in the airbag; retractable means (for example a cable or ratchet member) for extending in the airbag; fixing means for fixing the retractable means to the airbag; actuation means for repeatedly retracting the retractable means at least partially through the aperture to collapse the airbag, and for causing and/or permitting expansion of the airbag to an expanded state; and connecting means for connecting the actuation means to the airbag and for preventing egress of gas from within the airbag to the environment between the aperture and the actuation means.

The retractable means may be provided separately from the airbag and the fixing means may be for fixing by an operator.

Such apparatus overcomes the issues in the prior art, and enables, according to some embodiments, converting a conventional manual resuscitator into a fully functional automated ventilator. In some embodiments, the ventilator has patient monitoring capabilities.

Notably, U.S. Pat. No. 5,222,491A does not teach means for forming an aperture in an airbag, such as by piercing the airbag, or means providing an aperture in an airbag, for example by provision an annular piece with an openable closure. The former enables the apparatus to be used with conventional airbags. The latter enables attachment of other parts to the airbag. In contrast, the ventilator of U.S. Pat. No. 5,222,491A includes actuation parts permanently affixed to the airbag.

The apparatus may further comprise a housing for preventing gas from within the airbag from escaping to the environment via the actuation means.

The fixing means may comprise: means for penetrating the airbag to produce the aperture and a further aperture; and means for securing an end of the retractable means at the further aperture, wherein the securing means also seals the further aperture.

The penetrating means may comprise an elongate tool for producing the aperture and the further aperture in the airbag in a single penetrating action, wherein the tool and the securing means are configured to cooperate so that the tool can be used to pull the securing means through the aperture and an interior of the airbag, to secure the securing means in the further aperture.

The tool may be separable from the securing means.

The penetrating means may comprise a tool having a shaft having a sharp tip, wherein the tool is operable to produce the aperture and the further aperture in the airbag in a single penetrating action, wherein the securing means comprises first and second securing pieces, wherein the second securing piece is connectable to the first securing piece by location in a first hole through the first securing piece; wherein the connecting means comprises first and second connecting pieces, wherein the first connecting piece has a second hole therethrough, wherein the first securing piece and the first connecting piece are locatable on the shaft with the shaft extending through the first and second holes, wherein in the penetrating action the first securing piece locates in the further aperture and the first connecting piece locates in the aperture, with material of the airbag resiling to engage around the first securing piece and around the first connecting piece, wherein the second securing piece is attached to an end of the retractable means.

The first connecting piece, the second connecting piece and the actuation means may be relatively disposed when the first and second connecting pieces are connected so that the second securing piece is aligned with the first securing piece, wherein the first and second securing pieces are configured to connect in a press action.

The securing means may comprise a head and a neck, wherein the head and the neck are shaped such that material of the airbag around the further aperture resiles around the neck after stretching to enable passage of the head.

The fixing means may comprise a first fixing member mounted in the airbag, and a second fixing member mounted on an end of the retractable means, wherein the first and second fixing members are attachable by the operator.

The retractable means may be sufficiently rigid as to be pushed through the aperture, to push the air bag towards an expanded state, wherein the actuation means is configured to so push the retractable means, wherein the actuation means includes a controller and control of the actuation means to push or retract the retractable means is under the control of the controller.

Alternatively the retractable means may be flexible, and the fixing means may secure an end of the retractable means remote from the actuation means to the airbag. In this case the retractable means and an end of the retractable means near the actuation means are attachable to the actuation means.

The retractable means may comprise a line, wherein the actuation means includes means for drawing the line through the aperture and the opening.

The retractable means may comprise a ratchet member and the actuation means is configured with a gear for mating with the ratchet member to cause movement of the ratchet member.

The ratchet member may have a first, sharp end for penetrating the airbag to produce the aperture and the further aperture. In this case the fixing means may comprise a stop member located at an end of the rigid member remote from the sharp end, the stop means cannot pass through the further aperture produced in the airbag, and the rigid member is engageable with the actuation unit at a second end thereof.

The airbag may include: a first portion of resilient material, wherein the aperture is through the first portion; and a second portion of resilient material, wherein the fixing means fixes the retractable means to the airbag at the second portion.

At least one of the first and second portions may be weaker than portions of the airbag surrounding the at least one of the first and second portions, such that penetration is facilitated.

The apparatus may further comprise a controller operatively coupled to the actuation means, wherein the control means is configured to control the actuation means to draw the retractable means to collapse and/or expand the air bag wholly or partially.

The apparatus may further comprising a first user control coupled to the control means enabling user input of a valve for at least one variable, wherein the variable is at least one of: tidal volume, breathing rate, inhalation period, exhalation period, and wherein the control means unit is configured to control operation of the actuation unit in dependence on the at least one input value, where such operation includes control of at least one of: extent of retraction of the retractable means; rate of retraction of the retractable means; collapsing period; expansion period.

The apparatus may further comprise a sensor connected to the control means to provide a information thereto. In this case the control means is configured to control operation of the actuation means in dependence on information in the signal, wherein the sensor is locatable and configured to detect at least one of: information indicative of gas pressure within the airbag; information indicative of gas pressure within lungs of a patient; gas flow rate into the lungs; angular position of a drive shaft of a motor of the actuation means.

The sensor may be a pressure sensor located within the housing to detect pressure within the housing.

The sensor may be a pressure switch configured to provide information indicative of a predetermined threshold pressure being reached during collapsing of the airbag, wherein further to receiving the signal, the control means is configured to control the actuation means to cause or permit expansion of the airbag.

The control means may be configured to determine load on the actuation means, and to control the actuation means in dependence on the determined load.

According to a second aspect of the present invention, there is provided a method of setting up automated ventilation using an airbag, the method comprising: opening or forming by penetration an aperture in the airbag; fixing, by an operator, using fixing means, retractable means provided separately from the airbag to the airbag, so as to extend in the airbag; connecting an actuation means to the airbag, the actuation means when connected preventing environmental egress of gas from within the airbag at the aperture; causing operation of the actuation means to repeatedly retracting the retractable means at least partially through the aperture in the airbag to collapse the airbag, and to cause or enable expansion of the airbag to an expanded state.

According to a third aspect of the present invention, there is provided an airbag device of a ventilator, comprising: an airbag; a first connecting means mounting in wall material of an airbag, the first connecting means defining an aperture therethough and being connectable by an operator to a second connecting means of an automation unit, such that egress of gas within the airbag is prevented; a first fixing means mounted in the wall material of the airbag opposite the first connecting member, wherein the first fixing means is securable by the operator at an interior of the airbag to a second fixing means mounted on the end of a retractable means retractable by the actuation unit through the aperture.

The first fixing means may be securable by the operator to the second fixing means in an action whereby the first fixing means and the second fixing means are pressed together.

The airbag device may further comprise a removable closure member sealing the aperture, wherein when so sealed the airbag device can be operated to ventilate manually.

The airbag may have a first end at which a valve assembly is mounted for connection to a patient airway and a second end opposite to the first end, at which at least one valve is mounted for supply of gas to the airway, wherein the aperture is approximately midway between the first and second ends. The fixing means may also be arranged to fix an end of the retractable means within the airbag approximately midway between the first and second ends.

According to a fourth aspect of the present invention, there is provided apparatus for ventilation using an airbag, particularly for automating operation of an airbag of a manual ventilator, the apparatus comprising: an elongate ratchet member having a first end; an actuation unit including a gear configured to mate with the ratchet member to cause lengthwise movement of the ratchet member, wherein the actuation unit is configured to connect to a first portion of the airbag; fixing means for fixing the first end of the elongate member to a second portion of the airbag, wherein the actuation unit is operable to operate the gear to pull the second portion towards the first portion, thereby to collapse the airbag, and to control the gear to cause and/or permit expansion of the airbag.

According to a fifth aspect of the present invention, there is provided apparatus for ventilation using an airbag, particularly for automating operation of an airbag of a manual ventilator, the apparatus comprising retractable means for extending in the airbag; actuation means for repeatedly retracting the retractable means at least partially through the aperture to collapse the airbag, and for causing or permitting expansion of the airbag to an expanded state; and connecting means for connecting the actuation means to the airbag and for preventing egress of gas from within the airbag to the environment between the aperture and the actuation means; a housing for preventing gas from within the airbag from escaping to the environment via the actuation means; a control means, coupled to the actuation means for controlling the actuation means; a pressure sensor located within the housing, for providing pressure information to the control means; wherein the control means is configured to control the actuation means in dependence on the pressure information.

According to a sixth aspect of the present invention, there is provided apparatus for automating ventilation using an airbag, particularly for automating operation of an airbag of a manual ventilator, the apparatus comprising: an airbag;

retractable means (for example a cable or ratchet member) for extending in the airbag; fixing means for fixing an end of the retractable means to the airbag; a wall for sealingly separating the retractable means from a portion of the airbag containing gas for the patient; actuation means for repeatedly retracting the retractable means at least partially through an aperture in the airbag to collapse the airbag, and for causing and/or permitting expansion of the airbag to an expanded state; and connecting means for connecting the actuation means and the airbag.

According to a seventh aspect of the present invention, there is provided apparatus for automating ventilation using an airbag, particularly for automating operation of an airbag of a manual ventilator, the apparatus comprising: an airbag; retractable means (for example a cable or ratchet member) extending in the airbag; fixing means fixing a first end of the retractable means to the airbag; actuation means; means for securing the actuation means to a second end of the retractable means, wherein the actuation means is for repeatedly retracting the retractable means at least partially through an aperture in the airbag to collapse the airbag, and for causing and/or permitting expansion of the airbag to an expanded state; and connecting means for connecting the actuation means and the airbag.

In each of the sixth and seventh aspects, where the retractable means is a ratchet member, the member may be rigid or semi-rigid, such that the actuation means may cause expansion.

Also, In each of the sixth and seventh aspects, the airbag may have a first end at which a valve assembly is mounted for connection to a patient airway and a second end opposite to the first end, at which at least one valve is mounted for supply of gas to the airway, wherein the aperture is approximately midway between the first and second ends. The fixing means may also be arranged to fix an end of the retractable means within the airbag approximately midway between the first and second ends.

In each of the sixth and seventh aspects, the fixing means may permanently secure the end of the retractable means to the airbag. Alternatively, the fixing means may comprise a first fixing member mounted in the airbag, and a second fixing member mounted on an end of the retractable means, wherein the first and second fixing members are attachable by the operator.

### BRIEF DESCRIPTION OF FIGURES

Embodiments of the present invention will now be described, by way of example only, with reference to the accompany Figures in which:

FIGS. 1A and 1B are illustrations of a conventional ventilator with an airbag thereof in, respectively, a compressed state and an expanded state;

FIG. 2A is an illustrative, perspective view of an automation unit in accordance with an embodiment;

FIG. 2B is a close-up perspective view of parts of the automation unit shown in FIG. 2A, with a barb and a cable efflux portion shown spaced;

FIG. 3 is a block diagram indicating elements in the automation unit;

FIG. 4A is a cross-sectional view of parts of the automation unit attaching the actuation unit to fixation portions of an airbag;

FIG. 4B is a cross-sectional view of parts of the automation unit and the airbag, in a compressed state;

FIG. 4C is a perspective view of the airbag with the automation unit attached, after the airbag has been pressed so as to be pierced;

FIGS. 5A and 5B are respectively cross-sectional views of the airbag and parts of the actuation unit in partially collapsed and expanded states, with a cable spanning the airbag;

FIG. 6 shows illustratively user controls separate from a rest of the automation unit, coupled by a signal cable;

FIG. 7A is a cross-sectional view of parts of an automation unit in accordance with another embodiment;

FIG. 7B is a close-up perspective view of some of the parts of the automation unit shown in FIG. 7A;

FIG. 7C is a view of a tool shown in FIG. 7A;

FIG. 8 is a view of the automation unit including the tool shown in FIG. 7A, with a leading end of the tool shown detached as well as attached;

FIG. 9A is a view of an airbag including some integral automation parts, and an automation unit, in accordance with another embodiment;

FIG. 9B is a view of parts of the automation unit shown in FIG. 9A;

FIG. 10 is a view of the airbag shown in FIG. 9A;

FIG. 11 is a view of the airbag shown in FIG. 10, except with a closure for an aperture removed;

FIG. 12 is a view of an automation unit in accordance with another embodiment of the invention;

FIG. 13 is a cross-sectional view of an automation unit and an airbag in accordance with the embodiment shown in FIG. 12;

FIG. 14 is a cross-sectional view of an airbag, including some integral automation parts, and an automation unit, in accordance with another embodiment;

FIG. 15 is a cross-sectional view of an airbag, including some integral automation parts, in accordance with yet another embodiment;

FIG. 16 is an illustrative cross-sectional view of an airbag, including some integral automation parts. in accordance with another embodiment, wherein the automation parts into an elongate member having a toothed surface;

FIG. 17 is an illustrative cross-sectional view of the airbag and automation parts shown in FIG. 16, in a partially collapsed state that occurs when the airbag is collapsed manually;

FIG. 18 is an illustrative cross-sectional view of the airbag and automation parts shown in FIGS. 16 and 17, with a closure removed;

FIG. 19 is an illustrative cross-sectional view of the airbag and a ratchet shown in FIGS. 16 to 18 in a partially collapsed state that occurs when the airbag is automatically collapsed;

FIGS. 20A and 20B are illustrative cross-sectional views of parts of the actuation unit for controlling collapsing and expanding of the airbag shown in FIGS. 16 to 19;

FIG. 21 is an illustrative view of a ventilation device of the embodiment shown in FIGS. 16 to 21;

FIG. 22 shows illustratively how two ventilators each automated in accordance with an embodiment can be used with a junction device so that both provide air to a patient;

FIG. 23 shows illustratively that a single automation unit in accordance with another embodiment may be used with two ventilators so that both provide air to a patient;

FIG. 24 is an illustrative view of an airbag and an actuation unit in accordance with another embodiment in which the airbag is penetrated by a ratchet part of the automation unit;

FIG. 25 is an illustrative view of the parts shown in FIG. 24 when the automation unit is operated to partially collapse the airbag;

FIG. 26 is an illustrative view of the parts shown in FIGS. 24 and 25 when the automation unit is operated to collapse the airbag to the greatest extent possible;

FIG. 27 is an illustrative view of the parts shown FIGS. 24 to 26, where the automation device is operated to expand the airbag;

FIG. 28 is an illustrative view of an airbag including integral automation parts, where an elongate member having a toothed surface is contained within a collapsible sealed area separate from the internal portion of the bag.

FIG. 29 is a perspective view of a sleeve for an airbag in accordance with yet a further embodiment; and

FIG. 30 is a perspective view of an airbag with the sleeve wrapped around.

FIGS. 31A and 31B each show illustratively a tool with automation parts thereon, with material of an airbag being shown penetrated in FIG. 31B;

FIG. 31C shows illustratively an automation unit for use with an airbag after the automation parts have been transferred from the tool to the airbag;

FIG. 32A is a view of an airbag device with integral automation parts in accordance with another embodiment;

FIG. 32B is a perspective view of an automation device in accordance with the other embodiment, in an open configuration;

FIG. 32C is a cartridge for location in the automation device;

FIGS. 32D and 32D are perspective views of parts of the cartridge and a portion of the airbag device, where a fixing piece is aligned for insertion into an aperture in the airbag device;

FIGS. 32F and 32G are perspective views of the cartridge;

FIGS. 32H are 32I are respectively views of the automation device when in an open and closed configurations;

FIG. 32J is a view of the automation unit operatively attached to the airbag device;

FIG. 32K is a view of the automation unit with first and second fixing members engaged, but a rest of the airbag absent.

The Figures are illustrative only and scale is not necessarily the same in different figures of the same embodiment.

### DETAILS DESCRIPTION OF EMBODIMENTS

Like reference numerals are used to denote like parts herein.

Embodiments of the invention relate to automation apparatus for automating repeated collapsing of an airbag for ventilation. Embodiments relate in particular to automation apparatus for a manual ventilator, such as a bag valve mask (BVM) type ventilator, for positive pressure ventilation of a patient, to automate operation of the ventilator.

In some embodiments, the automation apparatus is comprised in an automation unit that is initially separate from the manual ventilator whose operation may be automated. In this case, the automation unit includes means for forming one or more apertures in an airbag of the manual ventilator, for example by piercing the airbag, and attaching to the manual ventilator. Such an automation unit may be used in some embodiments with conventional manual ventilators, provided the material of the airbag is such as to allow such one or more apertures to be formed.

In other embodiments, the automation apparatus includes an automation unit and automation parts provided integrally with the airbag, separately from the automation unit. The airbag is thus modified relative to conventional airbags to include such automation parts. For example, the parts may include an annular member having an aperture therethrough and located in a wall of the airbag so that the aperture provides an opening from an interior of the airbag to the exterior, and a quick release openable closure member closing the aperture. In this case, the automation unit is configured to cooperate with the automation parts included with the airbag to automate operation.

The automation apparatus may also include a tool to aid in coupling the automation unit and to the airbag.

The automation apparatus includes retractable means, such as a line in the form of a cable or a ratchet member, for extending through the aperture, the retractable means being fixed by fixing means to the airbag such that retracting the retractable means through the aperture collapses the airbag. The automation apparatus also includes actuation means operable to repeatedly draw the retractable means at least partially through the aperture to collapse the airbag and for facilitating expansion of the airbag to the expanded state. The automation apparatus further includes connecting means to couple the actuation means to the airbag and to prevent leakage of gas from within the airbag to the environment between the actuation means and the airbag. Although not essential, the connecting means may support a portion of the airbag to enable the airbag to be collapsed by retracting of the retractable means. Additionally or alternatively, separate countering means maybe provided.

In the embodiments to be described, the airbag comprises resilient material extending between first and second ends of the airbag to define an interior space. The aperture that is present or formed in the airbag is located approximately midway between the first and second ends, through the airbag material. However, embodiments of the invention are not limited to the aperture being provided or formed in a particular position. In some embodiments, the airbag is not part of a manual ventilator. Instead the airbag is for use solely with the automation apparatus to provide an automated ventilator.

In the embodiments to be described, the retractable means is fixed to the airbag material at one or more positions midway between the first and second ends, approximately midway between the first and second ends. In most of the embodiments, an end of the retractable means is fixed at a single position midway between the first and second ends and opposite the aperture. Embodiments of the invention are not limited to fixing the retractable means to any one or more particular positions within the airbag relative to the aperture, provided the retractable means can be drawn through the aperture to collapse the airbag and thereby to cause expelling of gas within the airbag through a patient connection valve assembly.

A portion of the airbag material around the aperture or one of the automation parts providing the aperture, depending on the embodiment, is herein referred to as the “first fixation portion” of the airbag material. Where an end of the retractable means is fixed to the airbag, a portion of the airbag material at which the end is fixed is referred to at the “second fixation portion”.

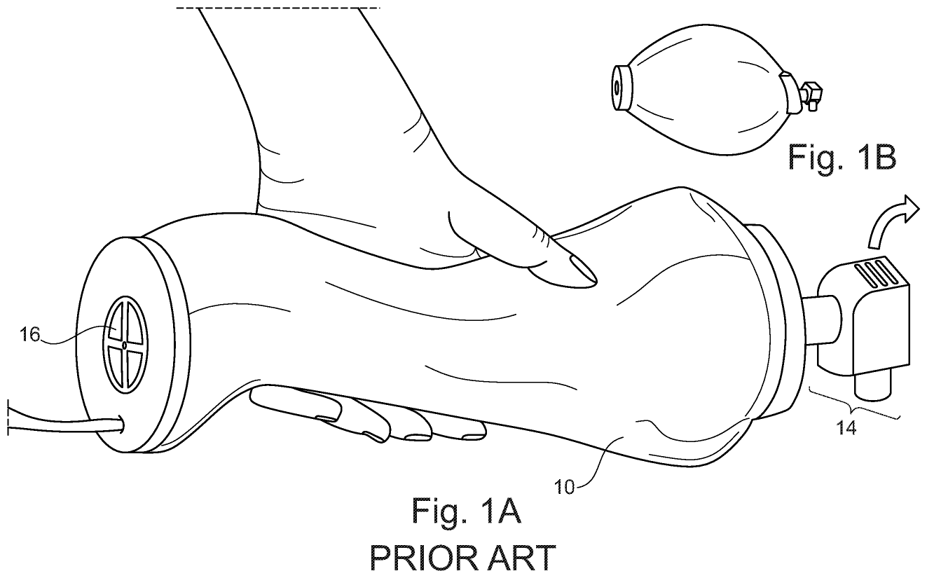

Referring to FIGS. 1A and 1B, a conventional ventilator comprises a resilient, compressible airbag 10, a gas source valve 16 and a patient connection valve assembly 14. The patient connection valve assembly 14 is connectable to an airway interface. The patient connection valve assembly 14 enables gas to flow into a patient's lungs by means of the airway interface and vents exhaled gas to the atmosphere.

The patient connection valve assembly 14 is operatively coupled to the airbag 10 at the first end thereof. The gas source valve 16 is operatively coupled to the airbag 10 at the second end thereof. The airbag 10 is compressible to a collapsed state when a compression force causes opposite sides of the airbag to move together. During compression, the airbag 10 expels air or gas in the airbag through the valve assembly 14. The airbag 10 is biased to an expanded state. Thus, when the airbag 10 is in the compressed state and the compression force is removed, the airbag 10 expands to the expanded state, drawing in air through the gas source valve 16. The gas source valve 16 may be configured to allow air from the environment to be drawn into the airbag 10 and/or may be connected to a gas source, for example an oxygen canister. In some of the Figures, the valve assembly 14 and the gas source valve 16 are not shown for ease of illustration. The manual ventilator described is representative of a conventional ventilator. In some embodiments, as already mentioned, the airbag is modified to include some of the automation parts. In this case, it should be understood that the airbag may be the same as the ventilator 10 except for modifications to accommodate those parts.

The airway interface may be a mask for location over the nose and mouth of the patient in a sealing manner. Alternative forms of airway interface may connect the valve assembly 14 to the patient, for example a tracheal tube or supraglottic airway.

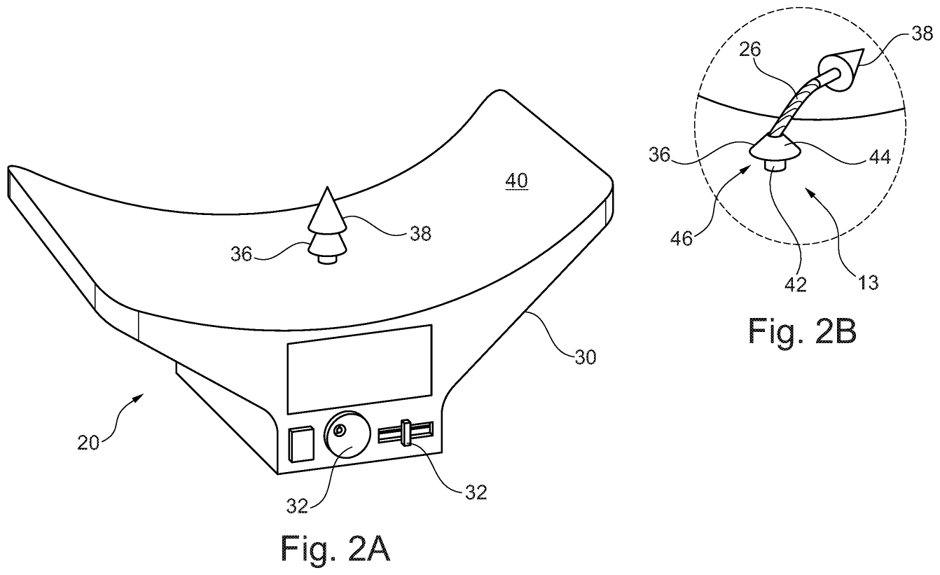

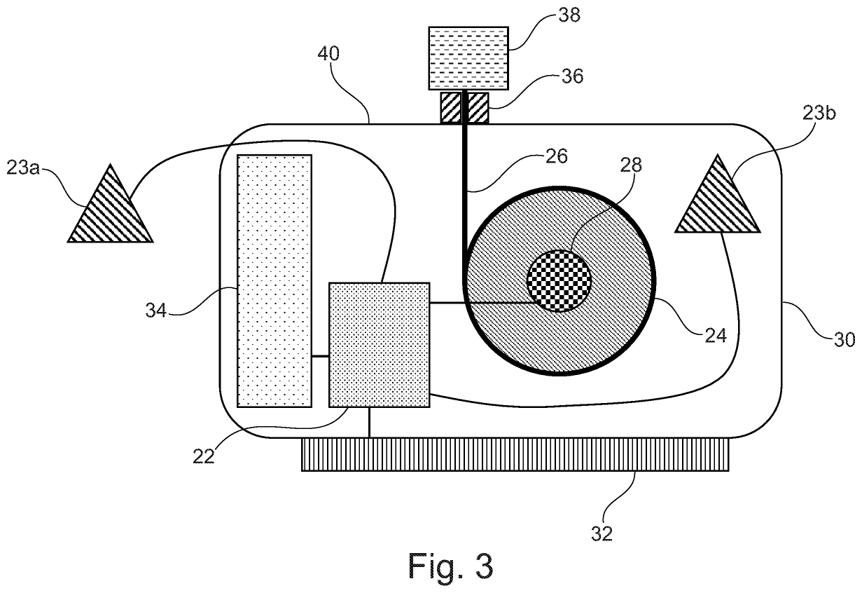

Referring to FIGS. 2A, 2B, 3, 4A, 4B and 4C, in an embodiment, an automation unit 20 is configured to attach to the airbag 10 of a manual ventilator, such as shown in FIG. 1A, so that the ventilator can be operated automatically. The automation unit 20 is configured to attach to a first fixation portion 13 of the airbag 10 and to a second fixation portion 15 of the airbag 10, the first fixation portion 13 being opposite to the second fixation portion 15. The automation unit 20 comprises a controller 22, a drawing means in the form of a take up reel 24, a retractable means in the form of a cable 26, an actuation unit in the form of an electric motor 28, a housing 30, user controls 32 and a power source 34. The automation unit 20 also includes a fixing means in the form of a fixation barb 38 fixedly mounted at a first end of the cable 26 remote from the electric motor 28. Optional sensors 23a, 23b, described in greater detail below, are coupled to the controller 22.

The cable 26 winds around the take up reel 24. The electric motor 28 is coupled to the take up reel 24 to cause winding of the take up reel 24 to draw in the cable 26 around the take up reel 24. The controller 22 is coupled to the electric motor 28 to cause operation thereof. The user controls 32 are coupled to the controller 22 and are operable by a user to provide control signals (e.g. start and stop) to the controller 22, for control of the electric motor 28. The user controls 32 may also enable the user to input values for predetermined variables.

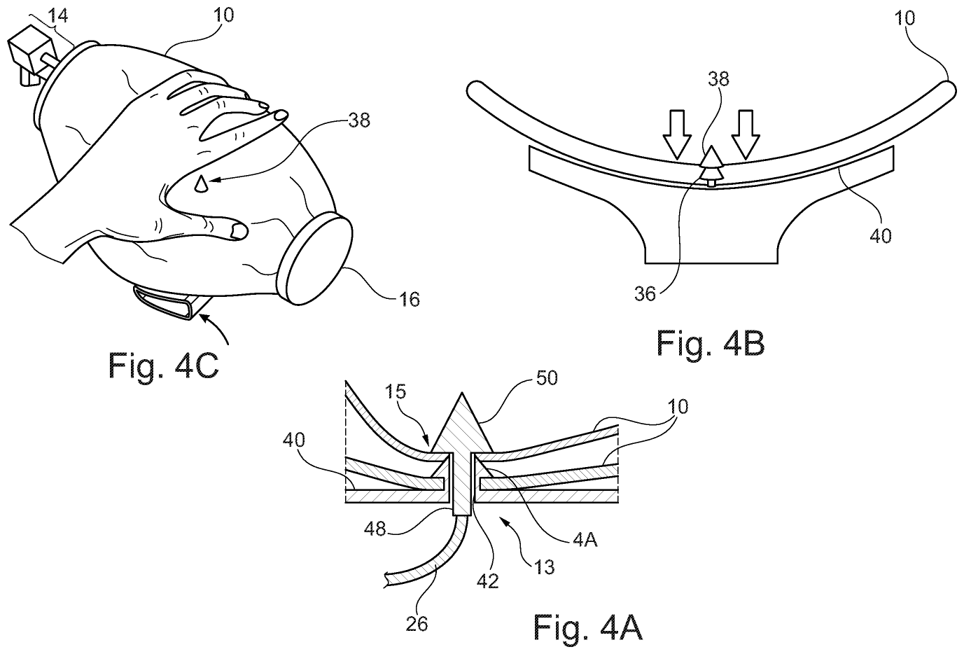

The unit housing 30 has an airbag support portion 40 and includes the connecting means in the form of a cable efflux port 36 in the airbag support portion 40. The airbag support portion 40 together with the cable efflux port 36 provide a counter to support the airbag during collapsing. Different variants of the airbag support portion 40 are illustrated in FIGS. 4A, 4B and 4C. The cable efflux port 36 provides a passage extending through the airbag support portion 40, through which the cable 26 extends in use. The cable efflux port 36 is configured to attach to the first fixation portion 13. Referring also to FIG. 4A, the cable efflux port 36 includes a neck 42 and a head 44, the neck 42 extending from the airbag support portion 40 and the head 44 extending from the neck 42. The passage extends through the neck 42 and the head 44 such that both extend circumferentially around the passage. The head 44 has a greater diameter than the neck to provide an annular recess 46 between the head 44 and the airbag support portion 40. The head 44 is tapered, having a surface sloping outwardly from an annular edge towards the airbag support portion 40.

The fixation barb 38 has a tip for penetration of the wall of the airbag 10. The fixation barb 38 also has a neck 48 and a head 50. The barb neck 48 is configured to locate in the passage of the cable efflux port 36. The barb head 50 tapers outwardly from the tip, such that it cannot be drawn into the passage of the cable efflux port 36, that is, the head 50 has base portion having a diameter that is greater than the diameter of the passage through the cable efflux port 36.

The fixation barb 38 is configured for penetration of the first and second fixation portions 13, 15 of the wall of the airbag 10 when the first and second fixation portions are pressed together and against the airbag support portion 40 of the unit housing 30. Since material of the wall is resilient, after penetration of the second fixation portion 15 by the fixation barb 38, material of the second fixation portion 115 resiles around the barb neck 48. Since the airbag 10 is biased into an expanded state, on stopping of the pressing the first and second fixation portions 13, 15 against the airbag support portion 40, the second fixation portion 15 moves away from the first fixation portion 13. The material of the second fixation portion 15 is configured to locate against the base portion of the barb head 50, as well as around the barb neck 48, in an airtight manner. The neck of the fixation barb is thick enough to ensure there is significant friction and a good seal between the fixation barb 38 and the neck.

The first fixation portion 13 is configured to resile into the annular recess 46 and to locate there in an airtight manner, such that the first fixation portion is fixed to the airbag support portion 40.

The controller 22 is in the form of a microcontroller having a processor and a memory. The memory has computer program code thereon. Execution of the code by the processor results in the functionality ascribed to the controller 22 herein. The controller 22 may be otherwise implemented, for example in hardware or a combination of hardware and software. The power source 34 is coupled to the controller 22 and the motor 28 for supply of power to the controller 22 and the motor 28. The power source 34 may be a battery, or may be a connecting lead and plug for connection to a fixed power socket.

The controller 22 is configured with configuration parameters in the memory thereof, including a rate at which the take up reel 24 is to be rotated for winding of the cable 26 around the take up reel 24. Since the airbag 10 has a predetermined maximum distance between the first fixation portion 13 and the second fixation portion 15, the rate of winding corresponds to a collapsing period in which the cable 26 is retracted to pull the first and second fixation portions 13, 15 of the airbag together from a maximum expanded state to a fully collapsed state. The controller 22 is configured to determine when the collapsing period has ended and to remove the collapsing force so that the airbag 10 can expand. The controller 22 may in some embodiments be configured to control the electric motor 28 to actively unwind, to encourage expansion of the airbag 10. It is not essential for the airbag to be collapsed to a fully collapsed state or to be permitted to expand to a fully expanded state. The controller 22 may determine amount of unwinding, for example in dependence on pressure information received from one or more pressure sensors.

Following expansion, the controller 22 is configured to restart collapsing by control of the electric motor 28 to wind the take up reel 24. Thus, the controller 22 controls the motor 28 to result in cyclical collapsing of the airbag 10 and thus provision of gas to the lungs of the patient, and expansion.

The configuration parameters may include any one or more of: the frequency of cycles of collapse and expansion; expansion period; and collapsing period. For example, the frequency may be one cycle every six seconds. The controller 22 is configured to control the motor 28 in accordance with the configuration parameters.

The user controls are coupled to the controller 22 for the operator to start and stop ventilation. The user controls may also be configured to enable input and/or modification of values for the configuration parameters. In embodiments to be described in the following, it is to be understood that the same automation unit may be used other than where it is clear that there are modifications.

In a variant embodiment, during expansion of the airbag 10, the cable 26 is kept under tension, but the tension in the cable 26 is insufficient to prevent expansion of the airbag 10. Such tension keeps the head of the fixation barb 38 pressed against an outer side of the second portion of the airbag 10, preventing egress of gas from within the airbag 10 through the aperture in the second fixation portion. Force applied to the cable 26 by the motor 28 during the collapsing period is sufficient to enable the winding at the predetermined rate, that is, to force gas within the airbag 10 to exit through the valve 14.

Preferably, the cable efflux port 36 is sized relative to the cable 26 so that gas in the airbag 10 does not egress through the aperture through the cable efflux port 36. Lubricant may be included to reduce any egress. Additionally or alternatively, the housing 30 seals regions of the interior of the automation unit, in particular around the take up reel, so that gas from within the airbag 10 cannot escape to the atmosphere via the aperture, and so the pressure of gas in the regions of the housing is substantially equal to that in the airbag 10.

In use, the airbag 10 is positioned by the operator so that the tip of the fixation barb 38 is pressed against an outer side of the first fixation portion 13 of the material of the airbag 10. The airbag 10 and the fixation barb 38 are then pressed together by pushing of the airbag against the airbag support portion 40, so that the fixation barb 38 penetrates, that is, pierces, the first fixation portion and then penetrates the second fixation portion, as indicated in FIGS. 4A and 4B. A pushing force is indicated with arrows in FIG. 4B. A hole created by the fixation barb 38 in the first fixation portion 13 facilitates pushing of the first fixation portion 13 over the head 44 of the cable efflux port 36, so that material of the first fixation portion 13 lodges around the neck 42 between the head 44 and the airbag support portion 40. As indicated in FIG. 4C, after being pressed against the airbag support portion 40, the fixation barb 38 is located against the second fixation portion 15 of the airbag 10. As indicated in FIG. 4B, the airbag support portion 40 may have a concave surface 40a to correspond to contours of the material of the airbag 10.

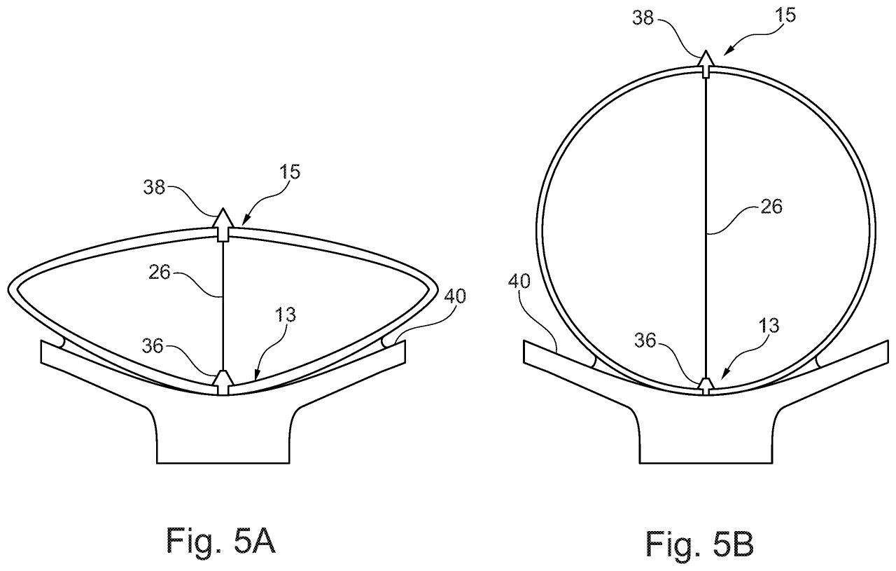

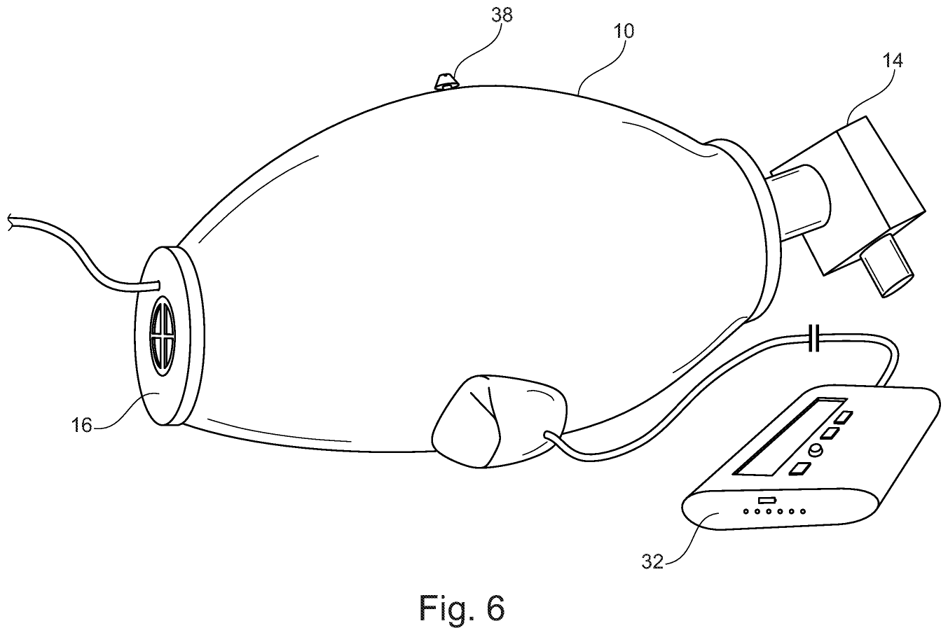

The controller 22 then controls the motor 28 in a cycle, dependent on the stored values for the configuration parameters and/or control values for the configuration parameters that the controller 22 determines to use for the particular patient. The controller 22 controls the motor 28 to allow extension of the cable 26 and the airbag 10 thus elastically recoils to an expanded state, as shown in FIG. 5B. When expanding, the airbag 10 draws in air or gas via the gas source valve 16. The controller 22 then controls the motor 28 to cause intake of the cable 26. This pulls the second fixation portion 15 of the airbag against the airbag support portion 40, thereby causing collapse of the airbag and forcing of gas within the airbag 10 into the patient's lungs. The partially collapsed airbag is illustrated in FIG. 5A.

In variant embodiments, the first fixation portion has an adhesive on an outer area thereof so that the airbag can be adhered to the airbag support portion 40. This helps the airbag 10 and the housing 30 stay in a wanted relative disposition in use. The adhesive may be a temporary adhesive.

Referring to FIG. 6, in an embodiment, the user control 32 is separate from other parts of the automation unit 10 and coupled to the other parts by a signal cable. The airbag support portion 40 is located flush against the airbag 10.

In some embodiments of the invention, the airbag 10 may be configured so that the first and/or second fixation portions 13, 15 are penetrable with less resistance than other portions of the airbag 10. To this end, in one example, the first and/or second portions are formed with less material than the other portions of the airbag 10. In this case, the first and/or second portions may be demarcated with visible indicia, such as a line, a visible change of texture or material, or a different colour to surrounding parts of the airbag.

Embodiments of the invention are not limited to any particular ways of fixing to the first and second fixation portions 13, 15 of an airbag. Alternatives to the fixation barb 38 may be used to fix an end of the cable 26 to the second fixation portion of the airbag.

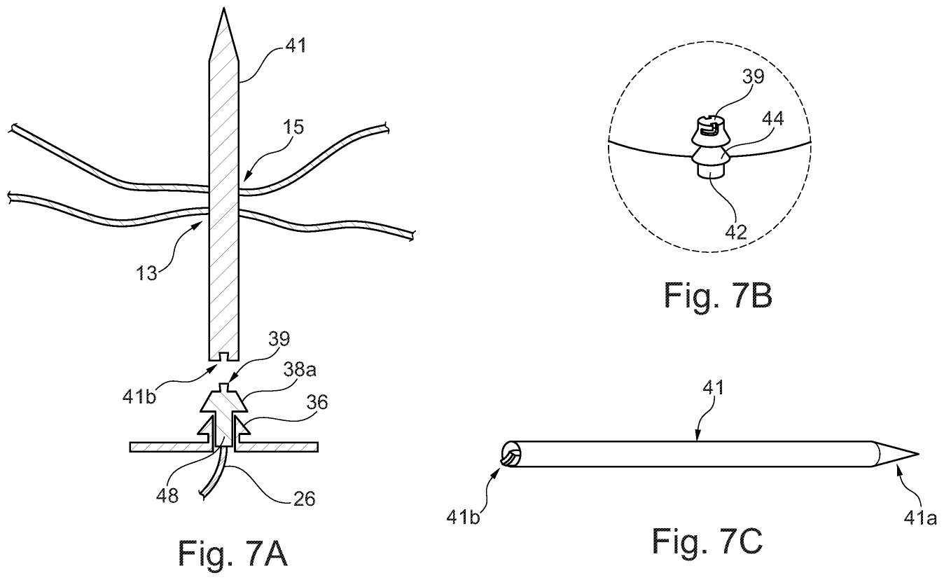

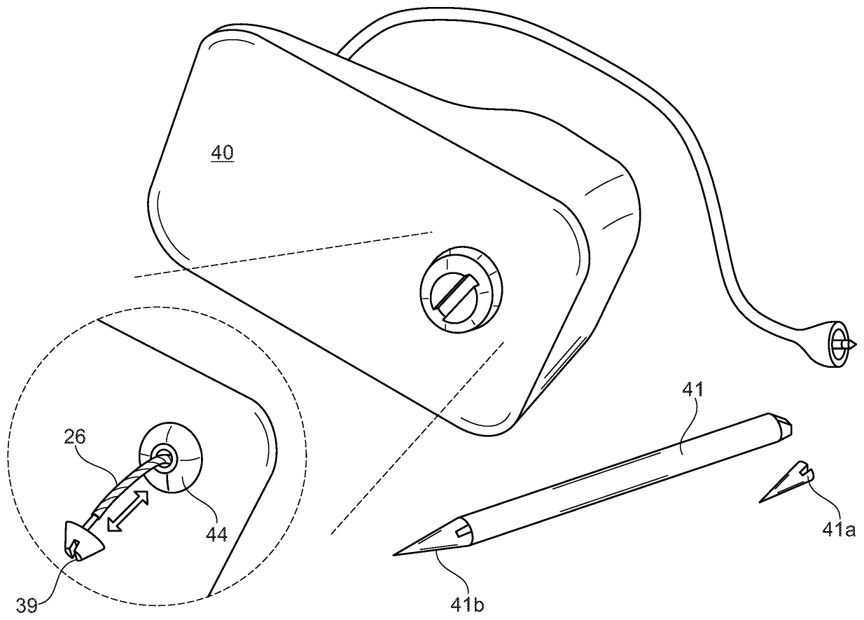

Referring to FIGS. 7A to 7C and 8, in an alternative embodiment, instead of using the sharp tip of the fixation barb 38 to penetrate the first and second fixation portions 13, 15, the automation apparatus includes an elongate penetration tool 41 to penetrate the first and second fixation portions 13, 15. In this embodiment, a fixation device 38a differs to the fixation barb 38 describes above in that in place of the sharp tip of the fixation barb 38 there is a first clip portion 39. The elongate penetration tool 41 has a sharp leading end 41a configured to penetrate both the first and second fixation portions 13, 15. A tail end of the tool includes a second clip portion 41b, configured to detachably attach to the first clip portion 39. In use, initially the first and second clip portions 41a, 41b are attached. An operator then locates the airbag 10 over the tool and presses the airbag onto the tool such that the tool 41 penetrates the first and second fixation portions 13, 15. The operator presses the material of the airbag over the tool 41 until, as described above, the first fixation portion locates around the neck of the cable efflux port 36 in an airtight manner, and the second fixation portion locates around a neck 48 of the fixation device 38a. The tool is then detached by unclipping of the first clip portion 39 from the second clip portion 41b.

In a variant embodiment, the first and second clipping portions 39, 41b may be absent. Instead, the tool 41 may be formed with the fixation device and be detachable therefrom in a snapping action. A portion of material between the tool and the fixation portion may be weakened relative to the rest of the material to enable snapping at that weakened portion.

In an alternative method of use, the tool 41 may be operated to penetrate the first and second fixation portions 13, 15 of the airbag 10 while detached from the fixation device 38a, with the first fixation portion 13 being penetrated by the leading end before the second fixation portion 15. The first and second clip portions 41b, 39 are then attached and the fixation device 38a is pulled by the tool 41 through the apertures produced in the first and second fixation portions. Such a tool 41 may provide an easy and convenient way to penetrate the airbag 10. The sharp end of the penetration tool 41 may be detachable or configured with a cover capable of protecting the sharp point during use.

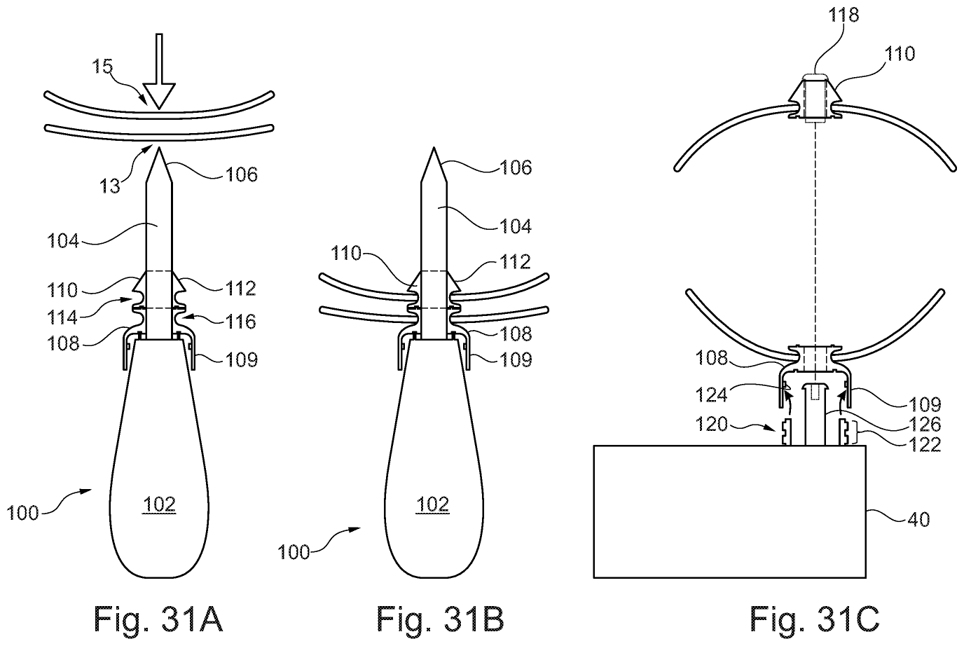

Referring to FIGS. 31A to 31C, in another embodiment, the automation apparatus further comprises a tool 100 comprises a handle 102, a shaft 104 and a sharp tip 106. The shaft 104 extends from the handle 102 and the tip 106 is at an end of the shaft 104 remote from the handle 102. The tip 106 is for piercing material of an airbag to produce an aperture in the first fixation portion 13 and a further aperture in the second fixation portion 15 of the airbag.

An annular mount 108 and a grommet 110 are both located on the shaft 104, with the annular mount 108 being between the grommet 110 and the handle 102. The grommet 110 has a tapered surface 112 and an annular channel 114 extending circumferentially around it. The annular mount 108 also has a respective annular channel 116 extending circumferentially around it. Surfaces of the grommet 110 and the annular mount 108 that are adjacent when the grommet 110 and the annular mount 108 are located on the tool 100 are flush. The grommet 110 and the annular mount 108 are slidable on the shaft 104.

The automation unit includes a grommet insert 118 at an end of the cable 26, indicated with dotted lines in FIG. 31C. The grommet 110 and the grommet insert 118 are configured to cooperate so that the grommet insert 118 can be pushed into a hole extending through a middle of the grommet 110, to clip securely therein and to form a seal for the hole, for example in a snap-fit manner.

The annular mount 108 also includes a socket portion 109 projecting away from the sharp tip 106. The automation unit includes a cylindrical projecting mount portion 120 slidable into the socket portion 109. The mount portion 120 includes outwardly facing first clip portions 122 and the socket portion 109 includes inwardly facing second clip portions 124. The first and second clip portions 122, 124 are configured to engage when the mount portion 120 is pushed into the socket portion 109 to provide an airtight seal between the mount portion 120 and the socket portion 109. The automation unit also includes a support 126 projecting from a housing of the automation unit within the mount portion 120 and having an internal passage through which the cable 26 passes. The support 126 projects from an opening of the mount portion 120. The support 126 is shaped so as to be located through the annular mount 108 when the mount portion 120 is engaged in the socket portion 109. The grommet insert 118 is initially located against an end of the support 126.

In use, the tool 100 is used by an operator to penetrate the material of the airbag in a single penetration action, with the sharp tip 106 being pressed through the material, to produce the aperture and the further aperture. The operator then continues to press the material onto the shaft 104 so that material at the first fixation portion 13 is pressed over the grommet 110 and into the annular channel 116 of the annular mount 108. The tapered surface 112 of the grommet 110 facilitates movement of the airbag material at the first fixation portion 13 onto the grommet 110, which the material then moves over onto the annular mount 108. The material resiles into the annular channel 116, forming an airtight seal between the channel 116 and the first fixation portion 13.

Pressing of the material onto the shaft 104 by the operator also presses the material at the second fixation portion 15 onto the grommet 110, where it resiles into the annular channel 114 so that an airtight seal is formed between the material of the airbag and the grommet 110. Again, the tapered surface 112 of the grommet 110 facilitates movement of the airbag material onto the grommet 110.

The tool 100 is then pulled away from the airbag, such that the sharp tip 106 is withdrawn. The grommet 110 and the annular mount 112 separate from the shaft 104 and remain in place.

The airbag, with the grommet 110 and the annular mount 112 secured thereto, is then connected to the automation unit, by locating the mount portion 120 into the socket portion 109 and thus clipping the first and second clip portions 122, 124 together. When this is done, an end of the support 126 extends through the annular mount 112 into the bag.

The grommet 110 is then pressed onto the grommet insert 118, so that the grommet insert 118 engages in the hole in the grommet 110. The automation unit is then operated to cause repeated drawing in and release of the cable 26, to cause repeated expelling of gas within the airbag.

Since the mount portion 109 and the socket portion 122 attach the airbag to the automation unit at the first fixation portion 13, the first fixation portion 13 is held in a fixed disposition relative to the automation unit. Thus, the mount portion 109 and the socket portion 122 provide a counter to enable collapse of the airbag by movement of the second fixation portion 15.

In this embodiment, the fixing means is in the form of the grommet 110 and the grommet insert 118, and the connecting means is provided by the annular mount 112, the mount portion 120 and the support 109 in place of the cable efflux port. Aside from these and from the tool 100, parts of the automation apparatus remain the same as previous embodiments.

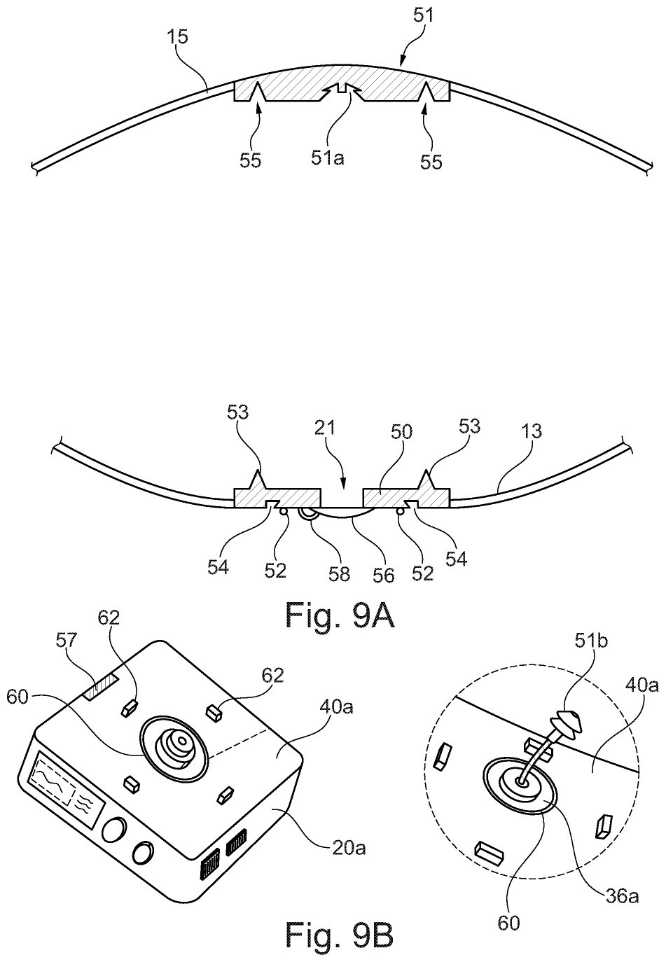

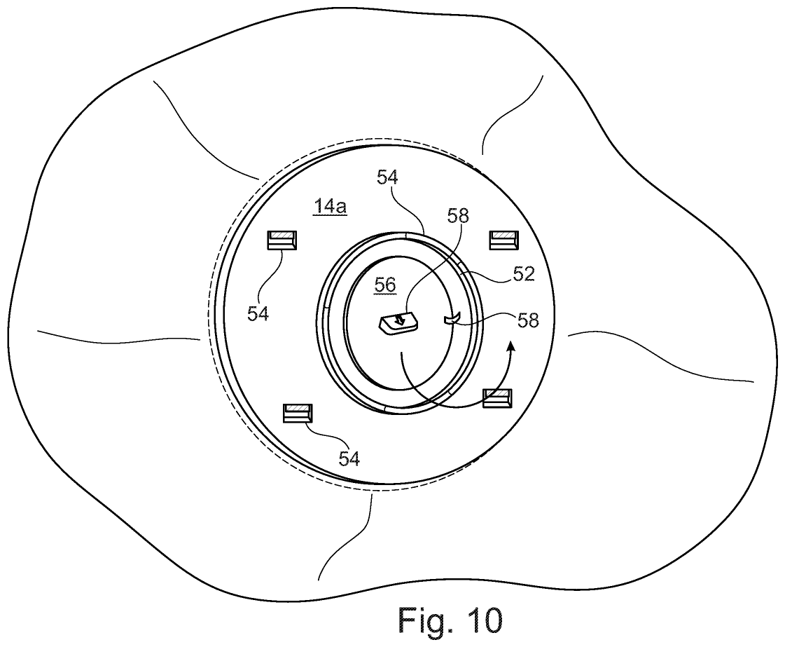

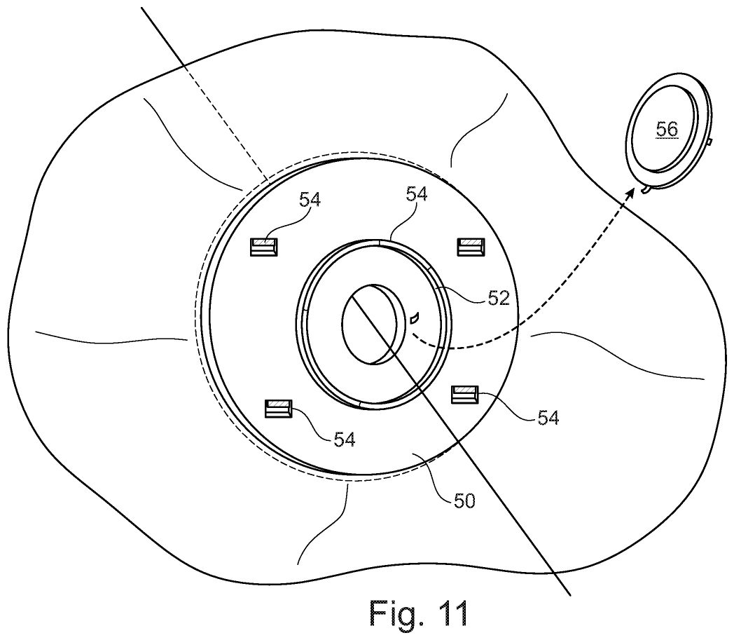

Referring to FIGS. 9A, 9B, 10 and 11, in another embodiment alternative fixing means and connecting means are used. In this embodiment, an airbag 110 is modified to include some of the automation apparatus. The automation apparatus incorporated in the airbag includes a rigid annular piece 50, for example of a plastics material, having a circular aperture 21 therethrough. Material of the airbag is fixed around the periphery of the piece 50 in an airtight manner, for example by glue.

The airbag includes a closure member in the form of a cover 56 secured to the annular piece 50 to close the aperture 21 in an airtight manner. When the ventilator is configured for manual ventilation, the cover 56 is secured to the annular piece to close the aperture 21. Referring to FIG. 10, the aperture 21 is openable by pulling of one or more projections 58 on the cover 56, which project to an exterior of the ventilator when the cover 56 is secured in place. In a variant embodiment, the annular piece 50 may be modified to have a threaded interior or exterior, and the closer member may be correspondingly threaded to screw onto the annular piece.

The connecting means for connecting the automation unit to the annular piece 50 comprises clip recesses 54 in the annular piece 50, spaced around the aperture 21, and a plurality of clip projections 62 spaced around the aperture 21 in the airbag support portion 40a. The clip projections 62 are configured to engage each in a corresponding one of the clip recesses 54. A button 57 is provided to release the clip projections 62 from the clip recesses 54 using a mechanism within the automation unit 20a (not shown).

In addition, the connecting means comprises an annular seal 52 projecting from the annular piece 50, the seal 52 extending around the aperture, and an annular groove 60 in the airbag support side. The seal 52 and the annular groove 60 are respectively configured so that the annular seal 52 can be pressed into the annular groove 60, to result in an airtight seal between the annular piece 50 and the airbag support portion 40a. Engagement of the clip projections 62 in the clip recesses 54 keeps the seal 52 pressed into the groove 60.

As will be appreciated, there are many alternative ways that the automation unit 20 could be modified to connect to the airbag, for example by screw attachment of the automation unit 20 to the annular piece 50, and/or to seal to prevent egress of gas from within the airbag to the environment between an exterior of the airbag and the automation unit 20.

A cable efflux port 36a is configured to fit within the aperture 21. Preferably (but not necessarily) the cable efflux port 36a is shaped to seal between the port 36a and the rigid annular piece 50, and thus differs from the port 36 in this respect.

In this embodiment the fixing means comprises first and second fixing pieces. The first fixing piece in the form of a rigid member 51, located in the second fixation portion 15 of the airbag, opposite the annular piece 50. In an alternative embodiment (not shown), the second fixing piece may be in the form of a rigid member secured to an interior surface of the airbag 10a, for example with an adhesive or by a mechanism means such as by riveting, or alternative chemical means.

The first fixing piece includes a first clip portion 51a. The second fixing piece is in the form of a second clip portion 51b and is attached to a first end of the cable 26. The second clip portion 51b is configured to engage with the first clip portion 51a in a snap fit manner when an operator presses them together.

Further, the annular piece 50 and the rigid member 51 respectively comprise an annular projection 53 and an annular groove 57, facing one another. The annular projection 53 and the annular groove 55 enable easy alignment of the annular piece 50 and the rigid member 51, to facilitate the operator engaging the first and second clip portions 51a, 51b together.

In use, the cover 56 is removed to open the aperture 21. The annular piece 50 is located against the airbag support portion 40a with the cable efflux port located in the aperture 21. The annular projection 53 and the annular groove 55 are then aligned and the operator then presses the rigid member 51 against annular piece 50, so that the first and second clip portions 51a, 51b engage. The automation unit is then operated to automate ventilation, as described above.

In variant embodiments, the first and second fixing pieces may be configured to otherwise engage. For example, each may be configured with suitable magnetic material for mutual attraction. In another variant, the first and second fixing pieces may be configured to adhere. In this case, the second fixing piece may include a surface with an adhesive on, and a releasable layer that the operator removes pressing the first and second fixing pieces together.

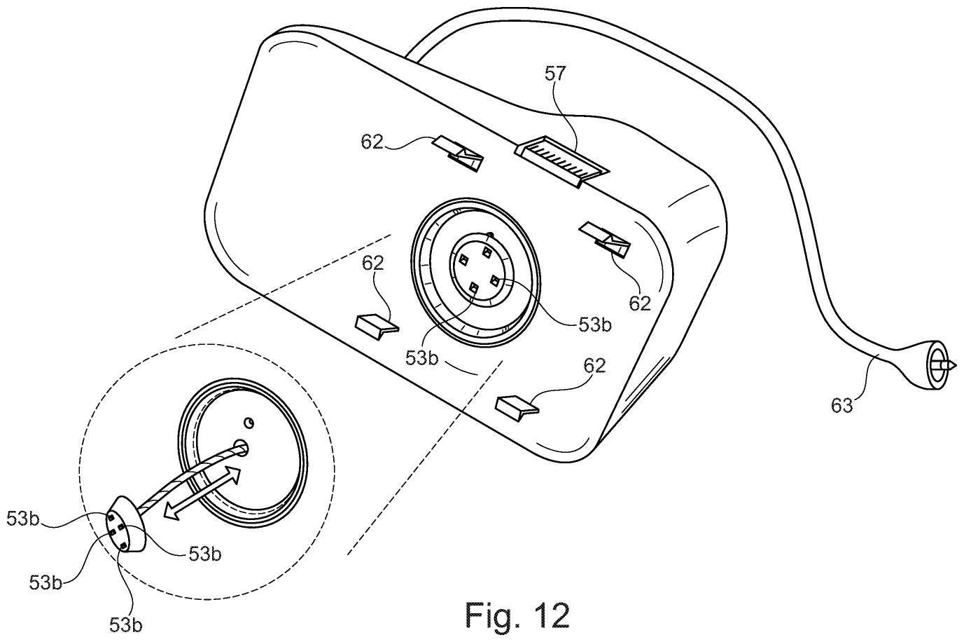

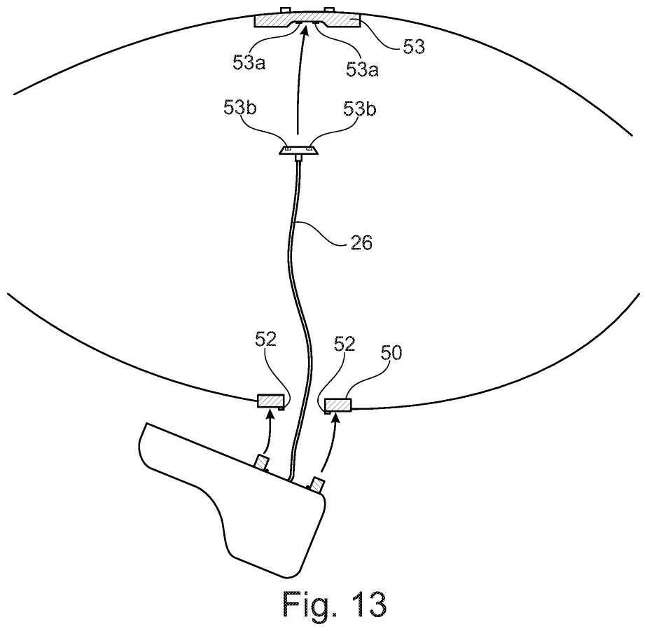

In another such variant embodiment, referring to FIGS. 12 and 13, in comparison to the embodiment described with reference to FIGS. 9A, 9B, 10 and 11, the first and second fixing piece differs; a rigid member 53 has a plurality of first clip portions 53a, and the second fixing piece 53b has a corresponding plurality of second clip portions 53b. In a pressing action by the operator, each first clip portion 53a is configured to engage with one of the second clip portions 53b. A cable 63 with a plug may be connected to a mains power source.

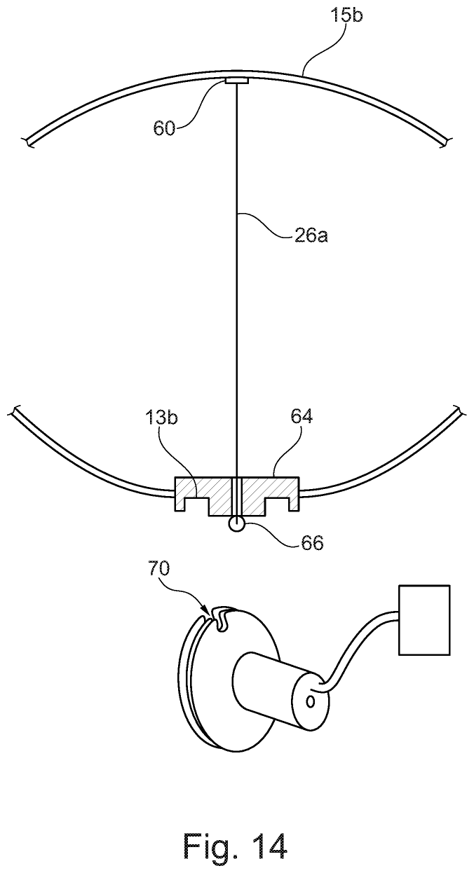

Referring to FIG. 14, in another variant embodiment, instead of a cable being initially secured around the take up reel, a cable 26a is secured within the airbag. A first end of the cable 26a has a mounting piece 60 secured thereto, which is fixed to the second fixation portion 15b with an adhesive. In variant embodiments, the first end may be otherwise secured to the second fixation portion 15b by mechanical and/or other chemical means. The first fixation portion 13b includes an annular piece 64 having an aperture therethrough, through which the cable 26a extends. The cable 26a has a bulbous portion 66 at a second end thereof located at an exterior of the airbag. The bulbous portion 66 and the aperture are respectively sized so that the bulbous portion 66 cannot pass through the passage.

The actuation unit in this embodiment includes a take up reel having a socket 70 configured to engage with the bulbous portion 66, so that operation of the take up reel draws in the cable 26 around the reel.

The bulbous portion 66 may be adhered or otherwise affixed to the annular piece 64 so that the airbag remains sealed during manual operation. The bulbous portion may be detachable from the annular piece 64 under finger pressure, or alternatively after engagement with the socket 70 the bulbous portion 66 may be sheared from the annular 64 when in the socket 70.

In use, the bulbous portion 66 is located in the socket 70. The take up reel is then controlled to draw in the cable 26a and to remove tension in the cable to allow expansion, as described above in relation to other embodiments. The take up reel is configured to attach fixedly to the annular piece 64, although details are not shown in FIG. 14, the actuation unit may connect to the annular piece 64 like in previously described embodiments, in a fixed manner to provide a counter to movement of the annular piece 64.

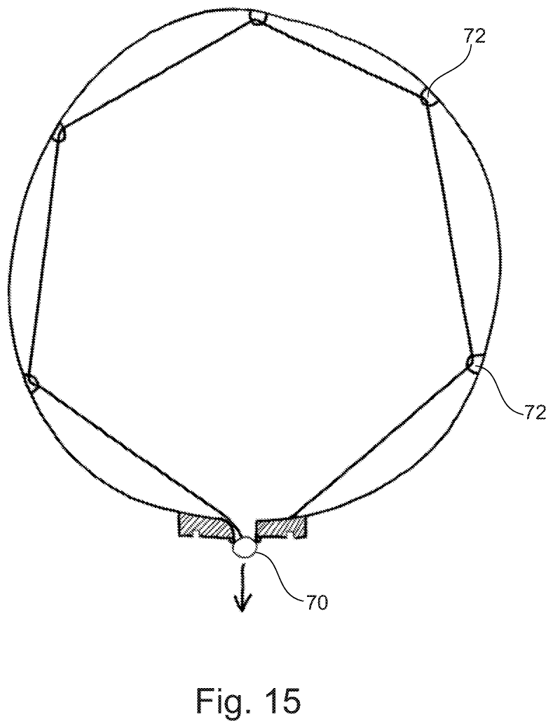

Referring to FIG. 15, in another embodiment in which a bulbous portion 66 is engaged by a socket 70, the airbag includes a plurality of second fixation portions, at each of which an eyelet 72 is mounted. The eyelets 72 extend around an interior surface of the airbag and the cable extends through them in a loop. In an alternative embodiment, the eyelets 72 may be mounted around an exterior surface of the airbag.

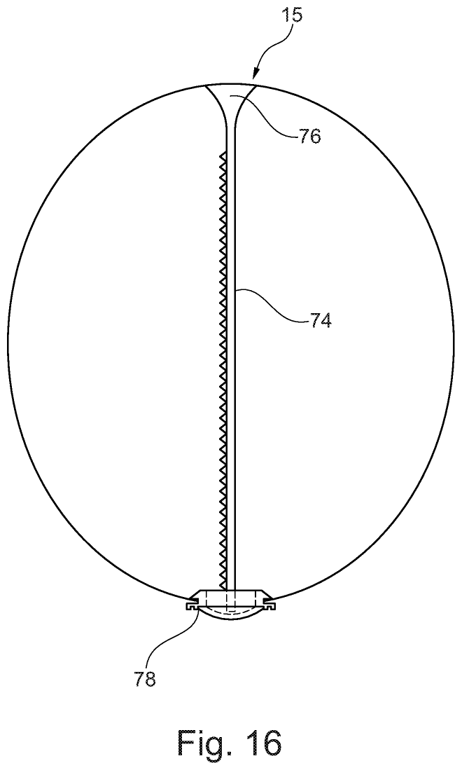

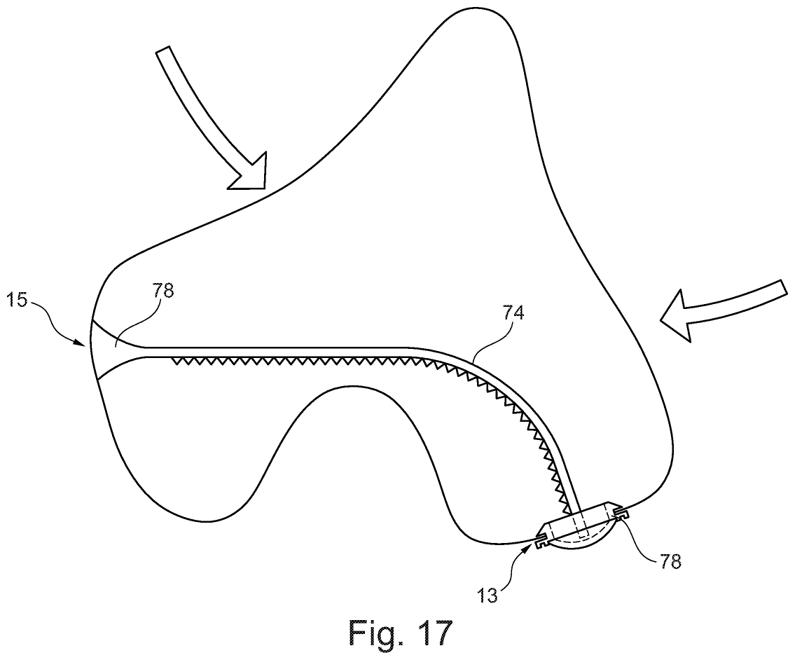

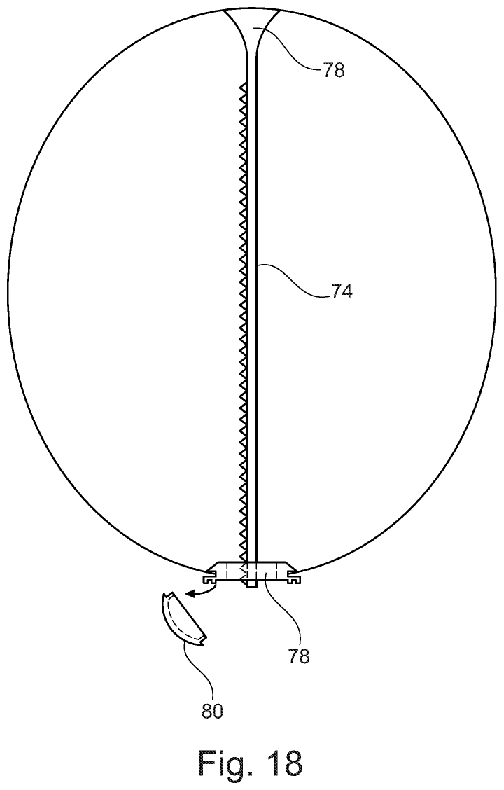

Referring to FIGS. 16 to 19, in another embodiment, instead of a cable 26 fixable at the or each second fixation portion of the airbag 10 and a take up reel, the retractable means is in the form of an elongate ratchet member 74 and the actuation unit differs in that it does not include a take up reel, but instead includes a retraction unit for retracting the ratchet member 74 through an aperture in the first fixation portion 13.

A first end of the ratchet member 74 is permanently fixed to the airbag at the second fixation portion 15 with fixing means in the form plastic mount 76 adhered to an interior surface of the second fixation portion 15. The first end may be otherwise fixed to the second fixation portion 15 in variant embodiments.

The automation apparatus includes a rigid annular piece 78 located the airbag at the first fixation portion 13c, providing the aperture, with the material of the airbag adhered or otherwise secured around it in an airtight manner. A second end of the ratchet member 74 is located at the aperture. The aperture is initially closed with a detachable closure member 80, so that the airbag can be used in manual ventilation. The ratchet member 74 is flexible, so that, when the closure 80 closes the aperture, the airbag can used for manual ventilation as indicated in FIG. 17.

Although connecting means for connecting the airbag to the automation unit is not shown in details in FIGS. 16 to 19, the rigid annular piece 78 and the automation unit may be coupled in the same way, using same features, as in the embodiment described with reference to FIGS. 9A, 9B, 10 and 11, or variants.

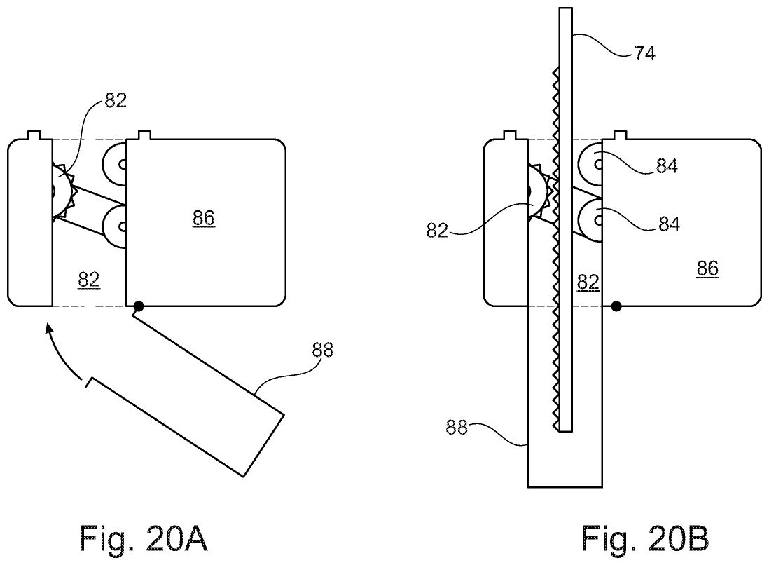

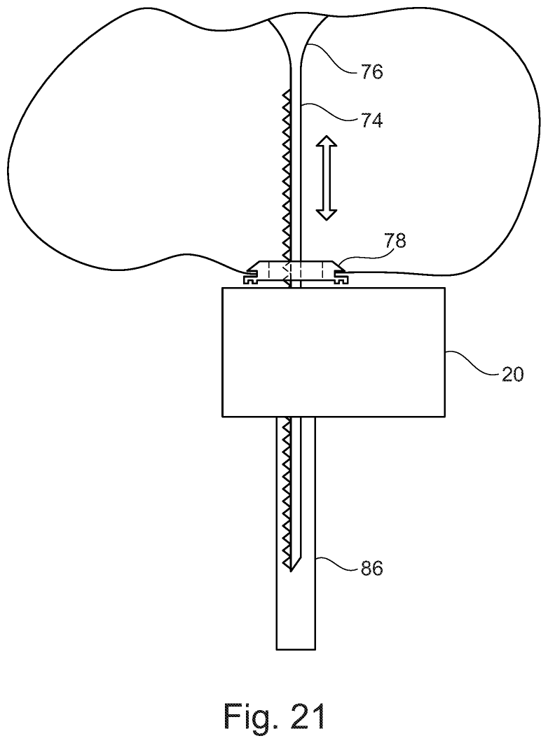

The ratchet member 74 has a toothed surface. Referring to FIGS. 20 and 21, the retraction unit comprises a gear 82, a pair of rollers 84 and housing 86. The housing 86 provides a passage through which the ratchet member 74 can be moved lengthwise. The gear 82 and the rollers 84 are mounted on the housing, so that the ratchet member 74 can be moved between the gear 82 and the rollers 84. The gear 82 meshed with the toothed surface of the member 74. The electric motor is coupled to the retraction unit to cause the gear 82 to rotate the gear 82, thereby to cause lengthwise movement of the ratchet member 74. The ratchet member 74 may be made of silicon, for example.

The retraction unit is attachable to the annular piece 78, to provide an airtight seal between an entry to the passage and the aperture. Referring to FIG. 21, the housing 86 includes a cover 88 for an end of the passage into which the second end of the ratchet member 74 extends in use. The cover 88 provides an airtight seal over the passage. The retraction unit is airtight, such that air cannot pass from the environment into the airbag.

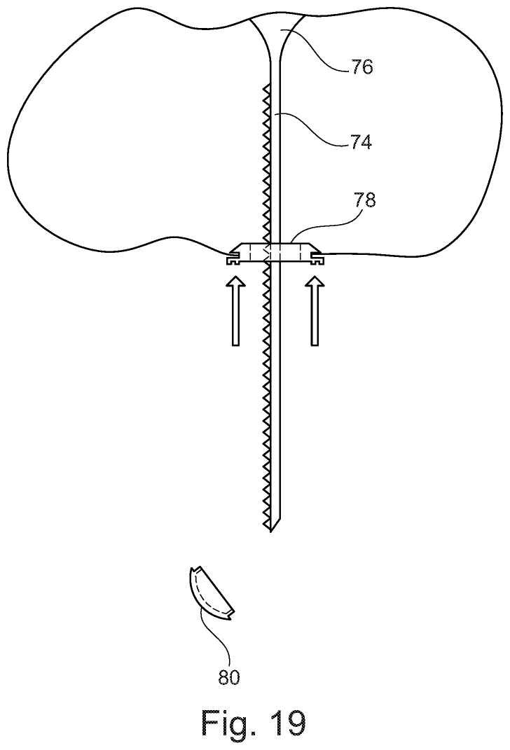

In use, the closure member 80 is removed from the aperture and the airbag is at least partially compressed by the operator so that the ratchet member 74 projects from the aperture (e.g. as in FIGS. 19 and 20). The second end of the ratchet member 74 is fed into the passage in the retraction unit. The motor 28 is then controlled by the controller 22 to cause the retraction unit to cause repeated extension and drawing in of the ratchet member 74. The retraction in of the ratchet member 74 causes collapse of the airbag and thus expelling of gas contained therein.

In some embodiments, after a collapsing period in which retraction has occurred, the controller 22 may control the motor 28 to allow the ratchet member 74 to move freely back into the airbag. Thus, when the airbag recoils to its expanded state, the ratchet member 74 is pulled back into the airbag. In some embodiments, the retraction unit is configured to control extension of the member 74 so that the rate and/or extent of expansion of the airbag is controlled.

Alternatively, instead of the ratchet member 74, the retraction means may include a piston member retained in a cylinder of the actuation unit, wherein the actuation unit is configured to cause extension and retraction of the cylinder under the control of the controller 22.

In variant embodiments, the ratchet member 74 may be sufficiently flexible to be retractable around a gear and the retraction unit may be configured accordingly with the take up reel 24 but having a gear.

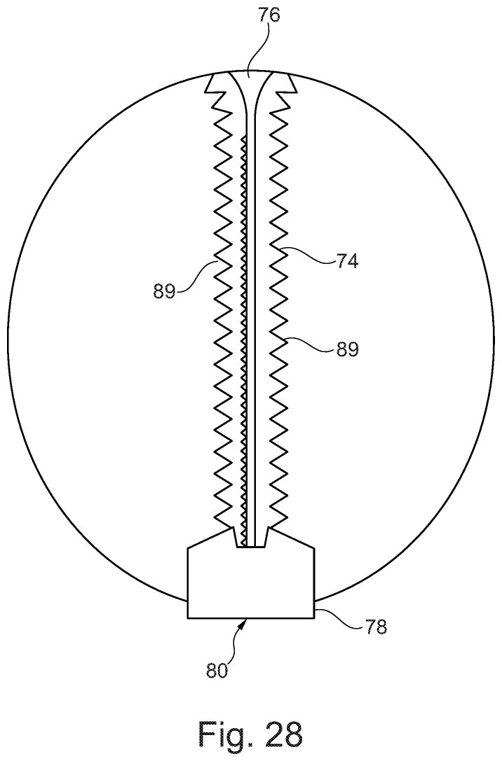

Referring to FIG. 28, the ratchet member 74 is located within the airbag in a portion of the airbag that is sealed from the region of the airbag that contains gas by walls 89. The second end of the ratchet member 74 may be accessed by detaching of the closure member 80 to open an aperture, as described above. Separating the ratchet member and the aperture from the region of the airbag that contains gas avoids need to have a good seal at the aperture to prevent egress.

Such walls may also be provided in embodiments in which the retractable means is in the form of the cable.

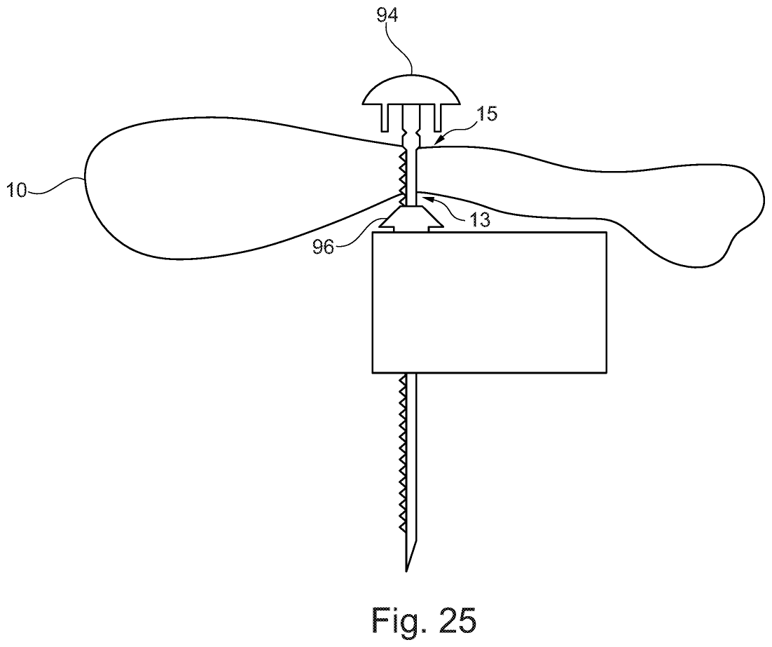

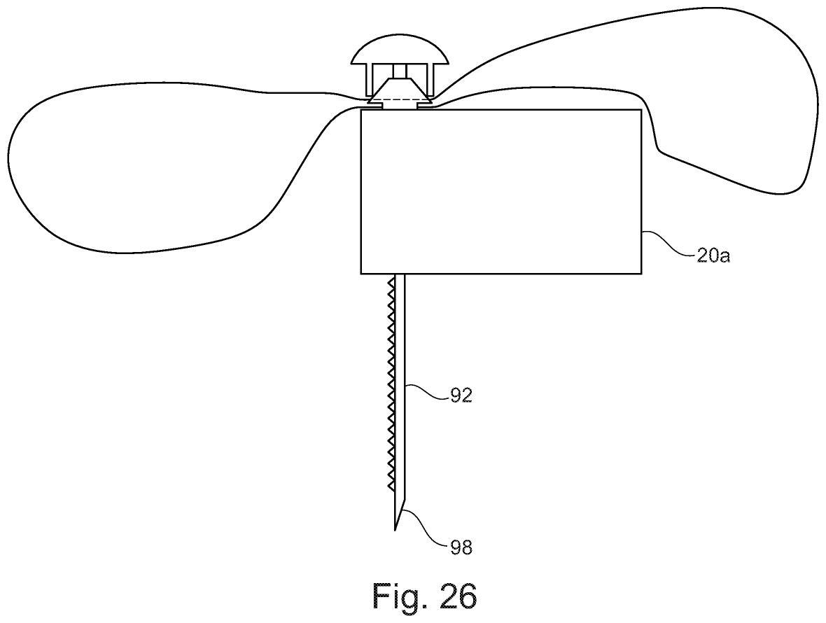

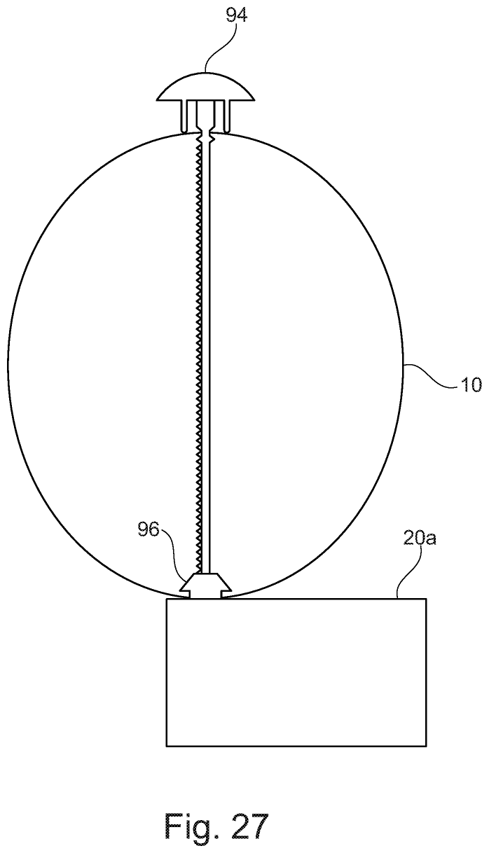

Referring to FIGS. 25 to 28, in another variant embodiment the automation apparatus includes a retraction unit for retracting a ratchet member like ratchet member 74, as described above, a toothed member 92, a fixing means in the form of a stop member 94 and a connecting means in the form of a resilient, annular seal 96. The toothed member 92 is rigid and a first end 98 is sharp. The stop member 94 and the toothed member 92 are formed of a single piece of material, for example a hard plastic, although this is not essential in embodiments. In variant embodiments, the stop member 94 and the toothed member 92 may be formed separately and secured together. The stop member 94 includes projections 95 to embed in the second fixation portion 15 to grip the airbag.

The seal 96 projects from the passage of the retraction unit to seal between the first fixation portion of the airbag and the retraction unit. The seal 96 has a mushroom shape and the material of the airbag 10, after been pierced and with the ratchet member located in the retraction unit, can be pressed over the head of the seal 96. The retraction unit can be then be controlled to control repeated expansion and collapse of the airbag. In a variant, the seal 96 may be adhered to the automation unit 20a. A release layer may be provided removable by the operator to expose pre-laid adhesive.

In use, the sharp second end 98 of the ratchet member 92 is used by an operator to penetrate first and second fixation portions 13, 15 of the airbag, such that the second end can be inserted into the retraction unit and the gear therein engaged with the toothed member 92. The stop member 94 prevents the first end of the toothed member 92 passing through the second fixation portion 15. In this embodiment, the actuation unit including the retraction unit is operable to retract the ratchet member 92 and also to extend the ratchet member 92. In this case, the retraction unit maintains a tension in the ratchet member 92, which results in the seal 96 sealing between the airbag and the automation unit, preventing egress of gas to the environment. In a variant embodiment, the connecting means may sealingly couple the automation unit to the airbag to prevent such egress, as described in relation to other embodiments. In this case, maintaining of tension in the ratchet member 92 is not essential.

In a variant to this embodiment, the toothed member 92 need not be rigid—only the sharp end need be hard. Instead the toothed member 92 may be flexible.

In the above described embodiments, several means by which an automation unit is coupled to an aperture in the first fixation portion 13 of an airbag have been described. Also airbag support sides 40, 40a have been described. Such parts are fixed disposed relative to the respective automation unit and provide a counter so that retraction of the respective retractable means through the aperture causes collapse of the airbag. In modifications, the airbag support portion 40 may be absent.

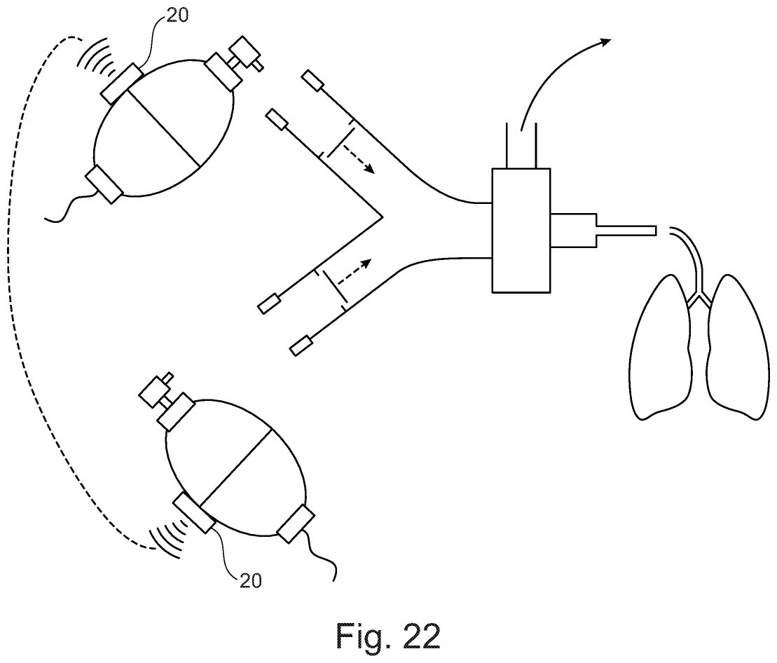

Referring to FIG. 22, in an embodiment two sets of airbags, patient connection valves, gas source valves and automation units are coupled to a junction device in which flows of gas expelled from the airbags are combined, to increase the amount of gas provided to a patient.

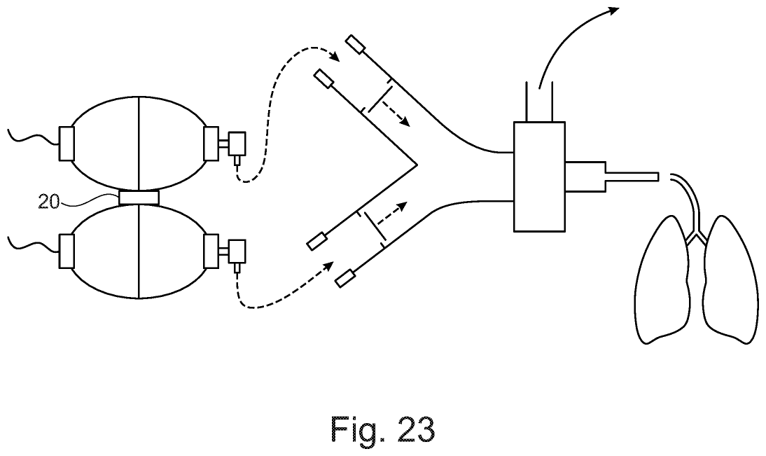

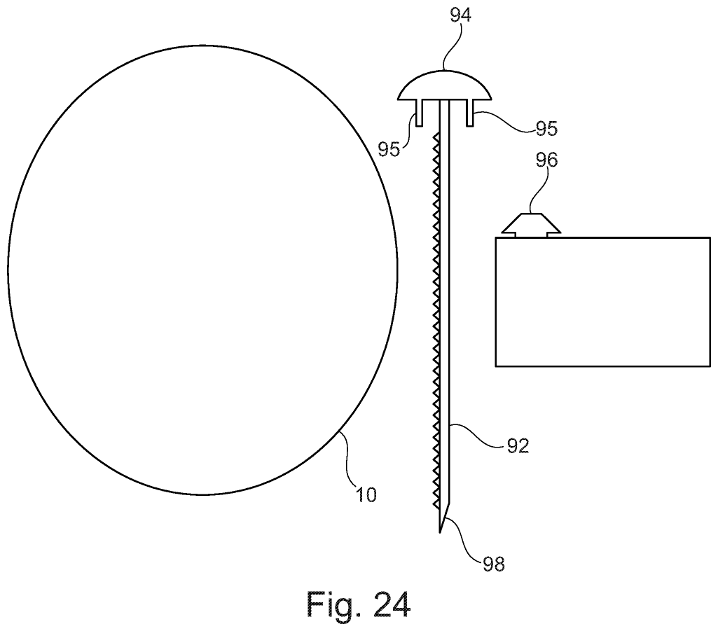

Referring to FIG. 23, in an embodiment expansion and collapse of two airbags is driven by a single automation unit adapted to control retraction of two opposing cables or two opposing ratchet members, depending on the corresponding design of other parts of the automation apparatus.

In the above described embodiments, gas from within the airbag can pass in use into the automation unit. The housing of the automation unit is configured to seal the unit to prevent egress of such gas to the environment and to prevent ingress of environmental air. The housing may include a portion sealing around the actuation unit, in particular around the take up reel or the retraction unit, depending on the embodiment.

A portion of the interior of the automation unit therefore includes gas at a pressure corresponding to pressure within the airbag. In an embodiment, a pressure sensor may be located in such a portion and be coupled to the controller 22 in a wired or wireless manner to provide information indicative of such pressure to the controller 22. In an alternative embodiments in which the automation apparatus is used with a conventional airbag, such a sensor may be mounted on the fixing means or the connecting means, such that gas pressure is detected within the airbag and indicated to the controller 22 in a wired or wireless way. In embodiments where the airbag includes parts of the automation apparatus, such a sensor may be mounted on one of the parts of the automation apparatus that comprise the fixing means or the connecting means where the sensor is exposed to gas at the interior of the airbag when the automation unit is connected.

In this case, a wire may be provided to connect the sensor to the automation unit and thus to the controller 22.

The embodiments described above may be modified so that the reel 24, in the case of a cable, or the retraction unit in the case of a ratchet member, may be permanently coupled to the airbag. In this case, a housing (rigid or flexible) may seal about these parts, with the cable or ratchet member located permanently in the airbag. In this case, other parts of the automation unit, such as the motor 28 and the controller 22, which tend to be more expensive of the parts, may be provided separately and used multiple times to automate operation of multiple ventilators.

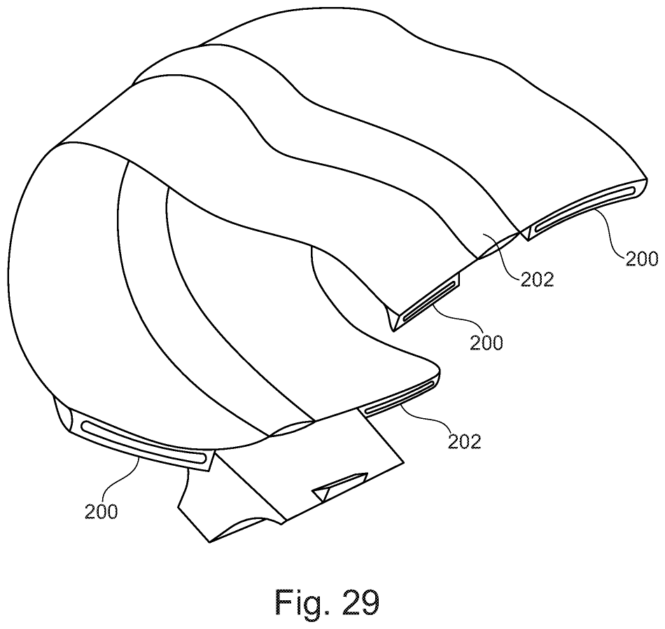

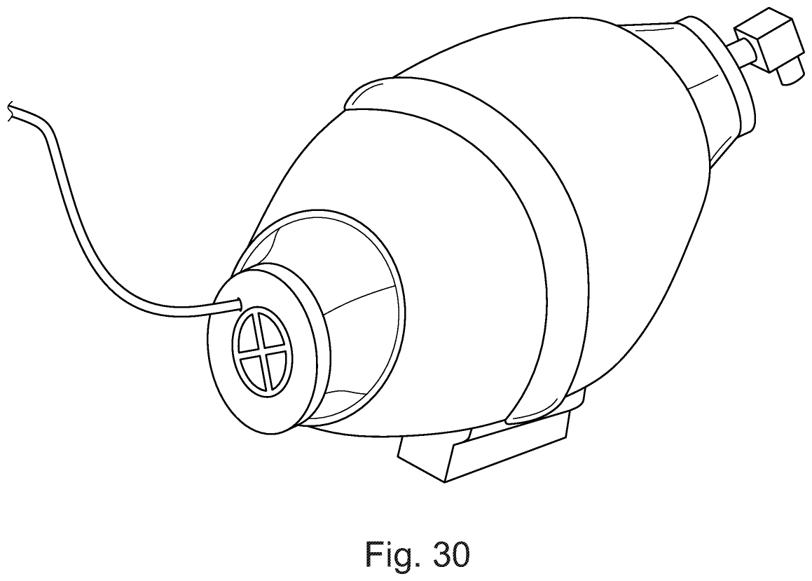

Referring to FIGS. 29 and 30, in another embodiment a sleeve is provided that may wrap around a conventional airbag. The sleeve includes a clip arrangement 200 for attaching ends of the sleeve. A passage housed by material 202 in the sleeve extends around the sleeve between the ends. A cable (not shown) extends in the passage and an end thereof is coupled to a take up reel of an actuator, as described above. The other end is fixed to the sleeve or has an end loop through which the cable extends. In use, the controller 22 operates to control the motor 28 to cause the reel 24 to draw in the cable, thereby to squeeze the airbag, and to release tension in the cable to allow expansion of the airbag.

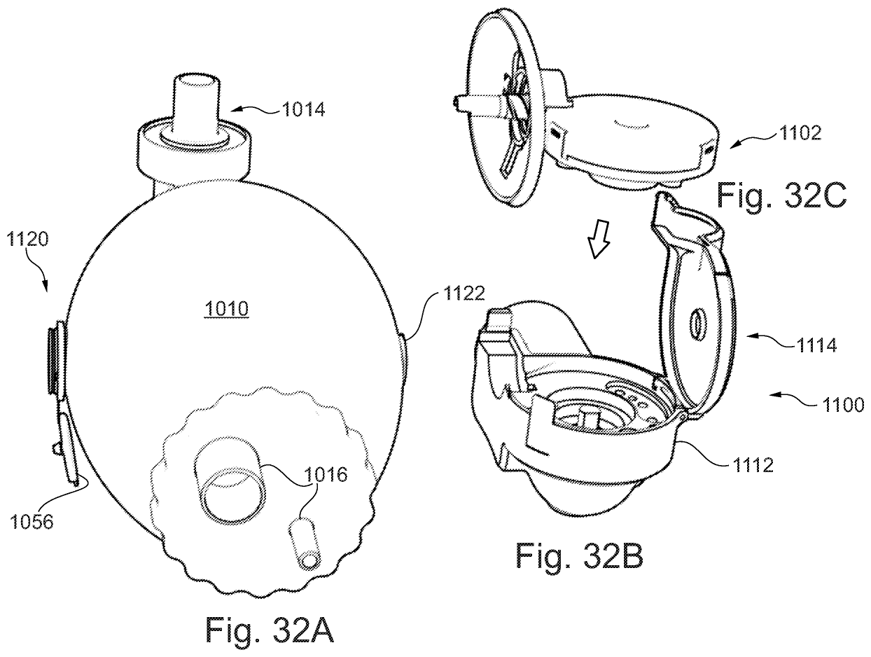

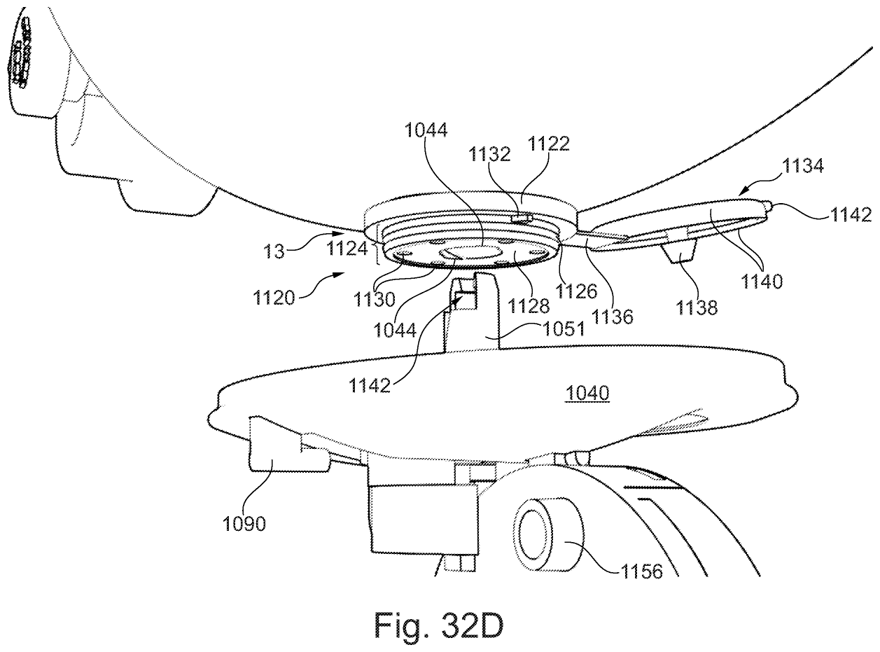

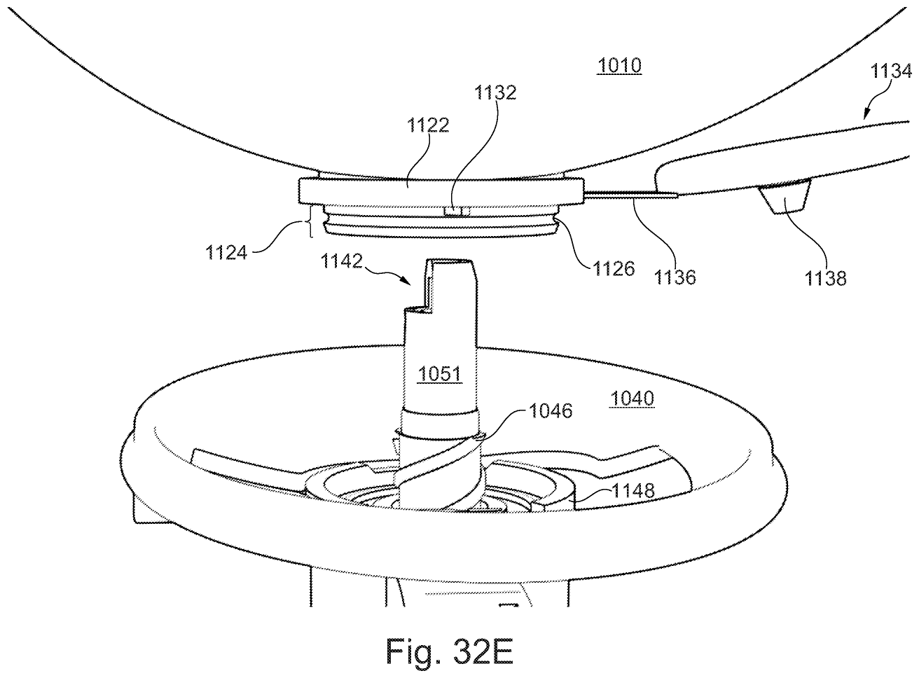

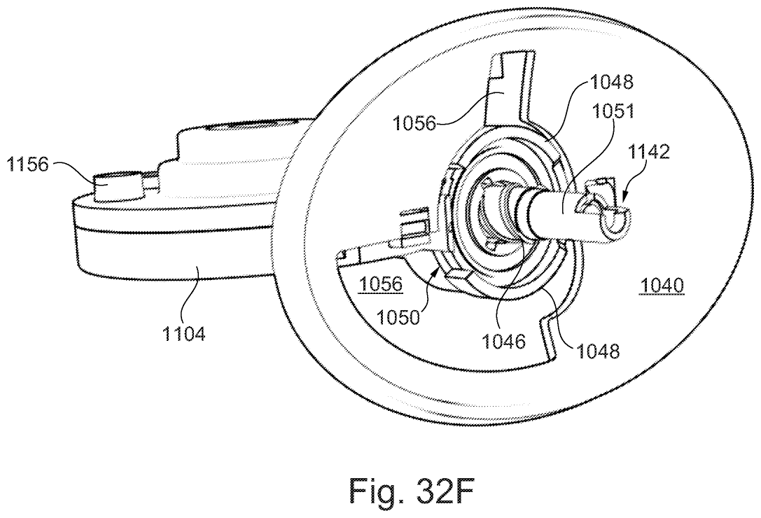

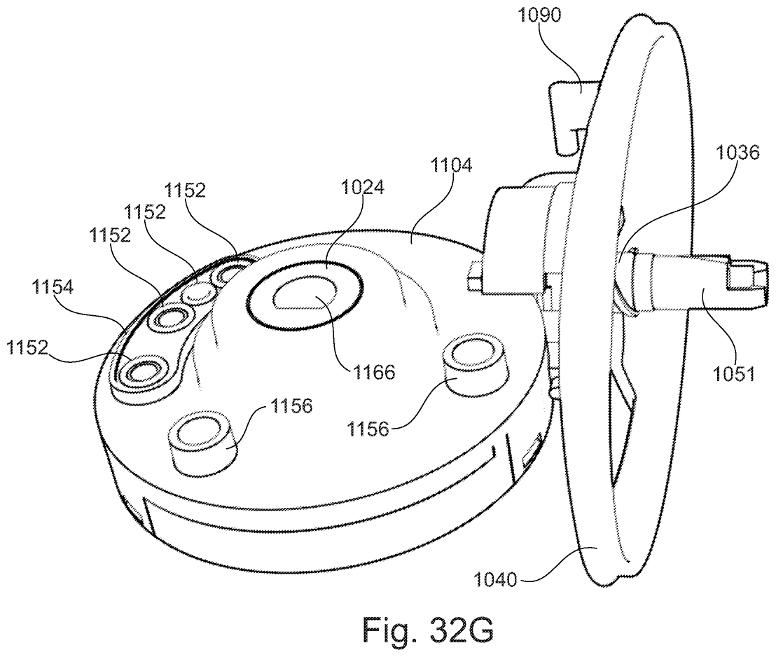

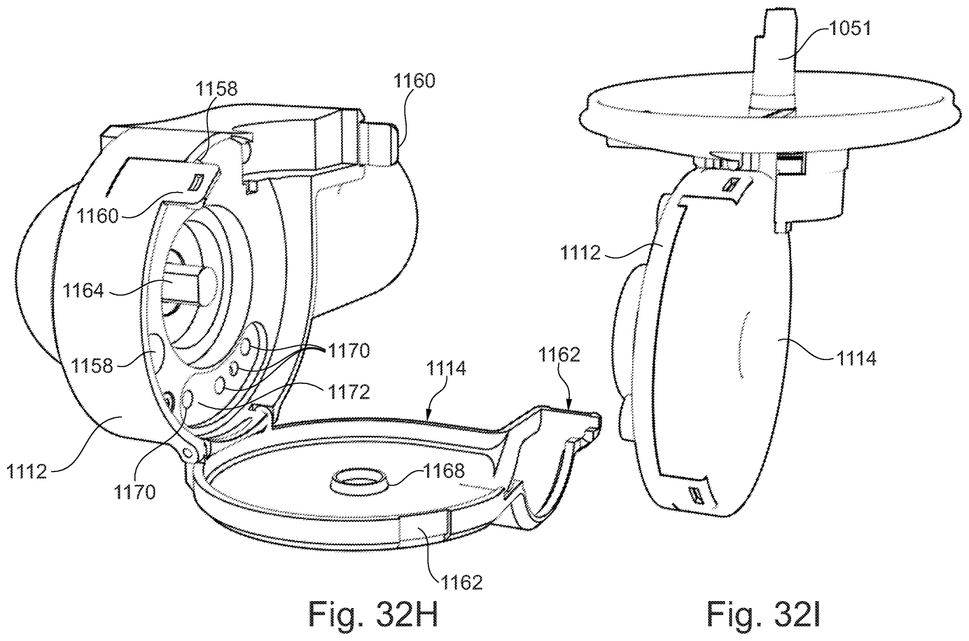

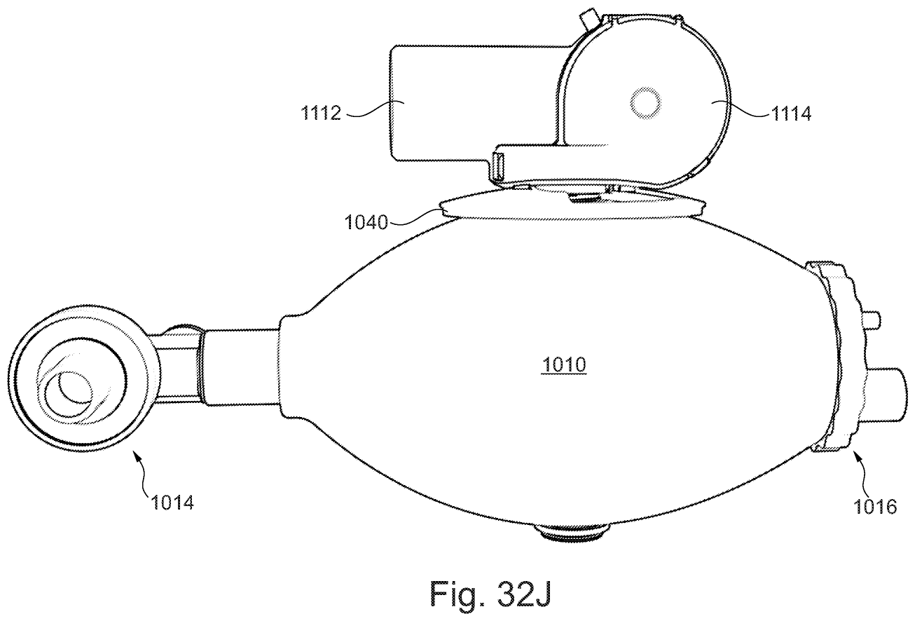

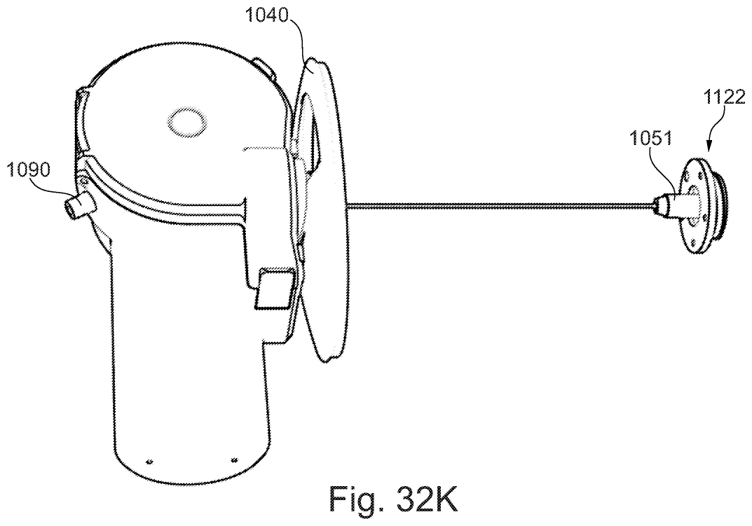

Another embodiment of the automation apparatus is now described in relation to FIGS. 32A-32J, the embodiment having features, including connecting means and fixing means, in common with the embodiment described above in relation to FIGS. 9A, 9B, 10 and 11. Parts that are functionally the same as parts in those Figures are indicated by the same reference numeral incremented by 1000.

The automation apparatus comprises an automation unit and automation parts comprised in an airbag 1010. Referring to FIG. 32A, a ventilator, which may be operated manually, comprises the airbag 1010, a patient connection valve assembly 1014 at a first end of the airbag 1010, and two gas source valves 1016 at a second end of the airbag 1010.

The automation parts of the airbag 1010 include a first connecting arrangement 1120 and a first fixing arrangement 1122. Referring to FIGS. 32B and 32C the automation unit comprises an automation device 1100 and a cartridge 1102. As better seen in, for example, FIG. 32G, the cartridge 1102 includes a housing 1104, a take-up reel 1024 and wound cable 1026, a cable efflux port 1036, a second fixing portion 1051, a second connecting arrangement, a countering means in the form of an airbag support portion 1040, and projections 1156. As will be described further, an end of the cable 1026 is attachable to the first fixing arrangement 1122 through an aperture in the first connecting arrangement 1120, and the second connecting arrangement renders the cartridge 1102 securable to the first connecting arrangement 1120. The automation device 1100 comprises a device housing 1112 and a closure 1114 hingedly mounted on the device housing 1112, as well as a controller (within the housing 1112) and an electric motor. The controller and the electric motor are functionally the same as those described in relation to all previous embodiments. After use with a patient, the ventilator parts shown in FIG. 32B and the cartridge 1102 may be disposed of, and the automation device 1100 may be used again with another such ventilator and cartridge 1102 on another patient.

Referring to FIGS. 32D and 32E, the first connecting arrangement 1120 comprises an annular piece having first and second rigid, aligned annular portions 1122, 1124 and a plate 1128. The first annular portion 1122 has a larger outer diameter than the second annular portion 1124, and is located in a first fixation portion 1013 of the airbag 1010 in a sealing manner, for example by glue or a fusing technique such as friction welding. The second annular portion 1124 has a circumferential channel 1126 around a periphery thereof. The plate 1128 is mounted on the second annular portion 1124 and extends inwardly with respect to the second annular portion 1124 to define an aperture into which the cable efflux port 136 can extend. The plate 1128 is screwed to the second annular portion 1124 with a plurality of screws 1130. The plate 1128 also defines the shape of the aperture. The second annular portion 1124 also has a lug 1132 projecting radially therefrom.

The first connecting arrangement 1120 also includes a closure member 1134 attached to the first annular portion 1122 by a flexible bridge 1136. The closure member 1134 includes a cylindrical side wall 1140 shaped to extend around the second annular portion 1124. Although not visible in the Figures, the cylindrical side wall has an inwardly projecting ridge shaped to engage in the channel 1126 in the second annular portion 1124 in a snap-fit manner, thereby to sealingly close the annular piece preventing egress of gas from within the airbag 1010. The closure member 1134 attaches to the second annular portion 1124 sufficiently strongly as not to pop off during manual use of the airbag 1010. The closure 1134 includes a tab 1142 to facilitate removal of the closure member 1134 from the annular piece by finger action of an operator.

The first connecting arrangement 1120 also includes a mushroom piece 1138 projecting from the closure 1134. When the closure member 1134 closes the annular piece, the mushroom piece 1138 projecting inwardly into the airbag 1010. The first connecting arrangement 1120 and the first fixing arrangement are, in the present embodiment, identical. The mushroom piece 1138 is not required for connecting of the cartridge 1102 and may be omitted. The two parts are identical so that the operator may connect the cartridge 1102 at either one. In variant embodiments it is not essential that these parts are identical.

While in a prototype the first connecting arrangement 1120 and the to first fixing arrangement were made from multiple separate parts, the first connecting arrangement 1120 may be manufactured as a single piece in an injection molding process.

The second fixing arrangement 1051 comprises a socket 1142 for the mushroom piece 1138. The mushroom piece 1138 is configured to engage into the socket 1142 in a snap-fit manner by pressure applied by the operator. Disengagement does not occur in use. The second fixing arrangement is fixed to an end of the cable 1026. The second fixing arrangement 1051 is initially located on the cable efflux port 1036, which is where it is drawn to when the cable 1026 is wound up around the take up reel.

The second connecting arrangement is configured to connect to the first connecting arrangement by a bayonet type connection. The second connecting arrangement comprises a pair of helical ridges 1046 projecting from an outer surface of the cable efflux port 1036 and extending from an end of the cable efflux port 1036 on opposite sides thereof towards the cartridge housing. As shown in FIG. 32D, the aperture has a circular part and a pair of recesses 1044 in opposing sides. The recesses 1044 are to receive the helical ridges 1046 and the cable efflux port 1036 is shaped to fit through the circular part of the aperture. When ends of the helical ridges 1046 are located in the recesses 1044, relative rotation of the airbag 1010 and the cartridge 1102 about a length of the cable efflux port 1036 results in the cable efflux port 1036 moving lengthwise into the airbag 1010. The cable efflux port 1036 is tapered so that the fit is tighter the more that the cable efflux port 1036 extends into the airbag 1010. The second connecting arrangement also includes a wall 1048 that extends from the cartridge housing around the cable efflux port 1036 with a gap 1050. The wall is shaped to extend around the second annular portion 1124. After relative rotation to a predetermined extent, the lug 1132 snaps into the gap 1150, the wall 1048 then preventing disengagement of the airbag 1010 and the cartridge and also preventing further relative rotation. The aperture and the cable efflux port 1036 are relatively sized such that the cable efflux port 1036 seals the aperture in an airtight manner when the lug 1132 is located in the gap 1050.

Although the first and second connecting arrangements connect together to seal with respect to the environment, gas from within the airbag 1010 may move into the cartridge 1102 through an interior of the cable efflux port 1036 and enter a chamber (not shown) provided by the cartridge housing 1104 that is sealed with respect to the environment other than by a plurality of holes 1152 that extend through the housing 1104. A sealing wall 1154 projects from the cartridge housing 1104 extending around the holes 1152.

The airbag support portion 1040 is shaped to provide support when the airbag is collapsed. The support portion 1040 is made of material that is sufficiently rigid to perform this function, while also being deformable for compact packaging. Holes 1153 are provided in the support portion 1040 facilitate the compact packaging.

Referring to FIGS. 32H and 32I, the automation device 1100 comprises a device housing 1112 and a lid 1114 hingedly mounted on the device housing 1112. The device housing 1112 is shaped to define a recess, the cartridge 1102 and the recess being shaped so that, when the lid 1114 is in an open position, the cartridge 1102 is locatable in the recess in a predetermined position relative to the device housing 1112. The device housing 1112 provides a pair of holes 1158 into which the projections 1156 can be located—when the projections 1156 are in said holes 1158 the cartridge 1102 is in said predetermined position in the device housing 1112. The lid 1114 is shaped to be closable over the cartridge 1102 and engage with the device housing 1112 to lock the cartridge 1102 within the automation device 1100. To this end, the device housing 1112 includes projecting clips 1160 and the lid 1114 includes clip engagement portions 1162. When the lid 1114 is closed against the device housing 1112, the clips 1160 engage in the clip engagement portions 1162 in a snap fit manner.

The device housing 1112 houses the electric motor, of which a drive shaft 1164 projects in to the recess for the cartridge 1102. As can be seen in FIG. 32G, the take up reel 1024 has a hole therethrough. When the cartridge 1102 is located in the automation device 1100, the drive shaft 1164 extends in the hole and engages with the take-up reel such that rotation of the drive shaft 1114 drives rotation of the take-up reel. To this end, the cross-sections of the hole 1166 and the drive shaft 164 are non-circular and shaped to mate. The lid 1114 includes a cylindrical portion 1168 projecting so as to receive an end of the drive shaft 1164.

The automation device 1100 includes a plurality of pressure sensors 1170 in a depression 1172 in the device housing 1112. When the cartridge 1102 is located in the automation device 1100, the sealing wall 1154 extends into the depression 1172 sealing the air holes 1152 and the pressure sensors 1170 with respect to the environment. The air in the vicinity of the pressure sensors 1170 is in fluid communication with air in the airbag 1010. The pressure at the pressure sensors 1170 is therefore indicative of the pressure in the airbag. In a variant embodiment, one or a different number of pressure sensors 1170 may be provided. In the present embodiment multiple are provided for redundancy. Although not shown, there is preferably an o-ring seal located over the opening of each air hole 1152 in the cartridge sized to seal between the respective air hole and a corresponding one of the pressure sensors, so that each pressure sensor 1170 provides information indicative of pressure within the respective air hole 1152.

The pressure sensors 1170 are connected to the controller 1022. In addition, the automation unit includes a port 1090.

Another pressure sensor (not shown) may be located within the port 1090 or in the automation unit in fluid communication with the interior of the port. The port is configured to connect to an end of a tube, whose remote end is locatable in (or in fluid communication with) the patient airway interface. Pressure in the patient's lungs may be determined using this pressure sensor in place of the pressure sensors 1170.

Although not shown in the Figures, the motor includes a sensor providing a signal to the controller 1022 of the angular position of the drive shaft relative to the initial position. The angular position corresponds to the extent of collapse of the airbag 1010.

In operation, where operation of the airbag 1010 is to be automated, the operator initially inserts the cartridge 1102 into the automation device 1100, if the cartridge is not already in place. To this end, the operator unclips the clips 1160 from the clip engagement portions 1162, to open the lid 1114. The operator then inserts the cartridge 1102 such that the projections 1156 extend into the holes 1158, the drive shaft 1164 extends into the take up reel 1024 and the sealing wall 1154 is located in the depression 1172. The operator then closes the lid 1114 such that the clips 1160 engage the clip engagement portions 1162. The cable efflux port 1036 also projects from an opening in the device housing 1112.

Before or after the cartridge 1102 is inserted, the cartridge 1102 is connected to the airbag 1010. To this end, first the operator removes the closure 1134 from the annular piece using the tab 1142.

The operator then connects the first and second connecting arrangements. This is done by the cable efflux port 1036 being positioned by the operator so that the ends of the helical ridges 1046 locate in the recesses 1044. In this position, the second fixing arrangement 1051 is located within the airbag 1010. The operator then rotates the cartridge 1102 relate to the airbag 1010 about the length of the cable efflux port 1036 such that the cable efflux port 1036 engages into the aperture. After rotation by a predetermined extent, the lug 1132 snaps into the gap 1150 and the cartridge 1102 is thus locked to the airbag 1010. The cable efflux port 1036 also seals the aperture such that egress of gas within the airbag to the environment is prevented.

The operator then presses the mushroom piece 1138 of the second fixing arrangement on the opposite side of the airbag 1010 into the socket 1142, such that the mushroom piece 1138 engages into the socket of the first fixing piece. The airbag 1010 then expands naturally, drawing in gas and pulling the cable from the take up reel.

The operator then controls the automation device to begin operation of the motor, that is, rotation of the drive shaft 1164.

The microcontroller causes repeated rotation of the drive shaft 1164 to an extent that is either predetermined or determined by the microcontroller, in the same way as in previously described embodiments, to repeatedly draw in the cable and thus collapse the airbag 1010.

The controller 1022 receives signals from the pressure sensors indicative of pressure within the airbag and is controlled to control operator of the motor in dependence on the pressure signals.

In a variant embodiment, the pressure sensors 1170 may be located in the cartridge 1102, for example where the holes 1152 are located in place of the holes 1152. In this case, where the holes 1152 open to the pressure sensors 1170, an electrical contact may instead be provided for electrical contact with a corresponding electrical contact in the automation device 1100, located where the pressure sensors 1170 are located in the illustrated embodiment.

In any of the above described embodiments, the controller 22, 122 may be configured to control any of the following variables:
extent of retraction of the retractable means;
rate of retraction of the retractable means;
collapsing period;
expansion period;
tension in the retractable means.

In the last case, the controller 22 may be configured to permit only a maximum tension, such that pressure in the airbag does not exceed a maximum.

The control may be dependent on any one or more of:
input of values for particular parameters from a user;
information received from one or more sensors;
information from the motor 28 and its circuitry, that is, load on the motor and angular position of a drive shaft of the motor (whether provided by a sensor or otherwise).

The controller 22 may be configured with algorithms stored in the memory thereof that use the received values and information to control the actuation unit, either in real-time, such that one cycle of retraction and expansion differs to the previous one, or in a fixed manner for the particular patient. As the skilled person would appreciate, various control architectures may be used to control the motor and the subsequent ventilation.

As indicated, in any of the embodiments described above, the controller 22 may control rate at which the retractable means is retracted and/or the extent to which it is retracted and/or a collapsing period over which the retractable means is retracted and/or an expansion period over which the airbag is permitted or caused to expand in dependence on the one or more values input by the user and/or in dependence on information received from the at least one sensor. The controller 22 may be configured to control said variables for a predetermined shape and/or size of airbag.

The automation unit may include circuitry enabling the load on/current drawn by the motor 28 to be provided to the controller 22. Such current is indicative of pressure in the airbag. Where the motor 28 drives retraction of the retractable means to at a particular speed, current drawn by the motor 28 will vary depending largely on the pressure within the airbag. A relationship between pressure in the airbag, the motor drive current and motor rotational speed may be used in control of the motor 28, for example to slow or stop retraction of the retractable means if the pressure is too high.

The motor 28 may include position sensing functionality (e.g. include a an angular position sensor providing information indicative of angular position of the drive shaft of the motor) and the automation unit may include circuitry including a sensor enabling position information to be indicated to the controller. The information is indicative of extent of rotation of the reel 24 or the gear 82 is available. Such information correlates to volume of gas expelled into lungs (tidal volume) and the controller 22 may control the motor 28 in dependence on a value for extent of rotation.

The user controls 32 may enable user selection of maximum current or voltage supply to the motor 28. In this case, during a breath of a patient, the controller 22 may be configured to control the motor 28 to retract the retractable means as fast as possible dependent upon the selected user selectable voltage and or current. Retraction continues until a maximum static load is achieved such that the motor is not able to continue to retract any further. The maximum load will correspond to a maximum peak pressure. As the voltage and or current is now at a maximum, power is reduced allowing passive recoil and re-expansion of the air bag ready for the next breath. Alternatively, the motor may re-wind until a fixed end stop position assisting the recoil of the bag.

In any of the above described embodiments, the airbag may include one or more sensors, such as a pressure sensor, or a gas flow sensor located in the air outflow port. The one or more sensors may be coupled or couplable to the controller 22 to provide information to the controller 22, and the controller 22 may control operation of the motor 28 in dependence on the received information.

Such a pressure or flow sensor may be positioned to measure pressure or flow closely approximating or matching that found within a patient's lungs. A conduit tube for transferring gas may extend from the automation unit to an adaptor piece located between an outlet valve connector and a patient airway interface. Alternatively, one or more sensors may be permanently located in the airbag and electrically connected to the automation apparatus with an electrical cable. Use of sensors allows real-time ventilation information to be provided to the controller 22. As mentioned above, the pressure sensor may be located in the automation unit in the portion at which gas pressure approximates pressure in the airbag.

The pressure sensor may be a binary pressure sensitive switch; such a switch and a mode of operation is described in patent publication no. WO2019016544, which is herein incorporated by reference in its entirety. The extent to which the airbag is collapsed may be controlled dependent on real-time information any one or more of these sensors. The controller 22 may also be configured to determine current used by the motor or position of the motor 28 in real time. As well as being any one of the sensors described above, each of the sensors 23a, 23b may also be in the form of pulse/heart rate monitor or device for measuring saturation of oxygen in the blood. The controller 22 may also or alternatively control operation of the motor in dependence on values for such parameters. For example if saturation is low the controller 22 may cause the motor to speed up breathing.

Where a pressure sensitive switch is provided, the switch is configured with a peak (threshold) pressure and to respond to pressure being above or below the peak pressure by sending a signal indicative of such to the controller 22. The controller 22 is configured to control operation of the motor to repeatedly cause deflation and allow inflation dependent at least on information in the signal. For example, the controller 22 may control the motor to control rate of ventilation and/or volume of gas expelled into a patient's lungs.

In a basic mode of operation, the pressure switch is configured to respond to pressure reaching the peak pressure by providing a signal to the controller 22. In response to the signal, the controller 22 is configured to control the motor to allow inflation of the airbag for a predetermined period of time. After that period of time has elapsed, the controller 22 is configured to cause the motor to collapse the airbag, until the switch indicates that the pressure has again reached the peak pressure. The peak pressure may be adjustable by the operator using the user controls. Additionally or alternatively, the breadth rate and/or expiration length may be set by the operator using the user controls.

Further, in any of the above described embodiments, the user controls 32 may include at least one control, for example switches or dials, for inputting values for parameters. The user controls 32 may allow input of information indicative of any one or more of: a volume of air to be forced into a patient's lungs; patient physiological observations, such as oxygen saturation; respiratory rate; inspiratory/expiratory ratio; peak pressure; pressure rise profile; and tidal volume.

Use of the above sensors allows rapid detection and understanding of clinically important parameters such as lung compliance, tidal volumes, airway resistance and circuit leak. If, for example, there was a slight leak in the system or if the patient's lungs are punctured, the bag would require faster compression to generate the equivalent pressure requiring increased motor torque. The controller 22 may be configured to compensate for clinical issues such as leak. If automatic compensation is not possible, an alarm may alert the responsible clinician. For example, if, during a breath, there is airflow detected and no meaningful pressure rise, it is likely a significant gas leak in the system is occurring thus preventing the build up of pressure. Alternatively, if the gradient of the pressure rise begins to significantly change, this likely indicates variable lung compliance. By combining real time pressure information from a pressure sensor with flow data, the calculation of exact lung compliance values may be determined. Derived clinical data may be displayed on an automation unit screen or transferred to a remote display or hardware such as a separate device that may also be capable of adjusting automation unit ventilation variables. However, as will be appreciated by the skilled person, detailed description of control architectures, which may depend on any one or more values received in real time from the sensors, as well as values input via the user controls 32, and also include various safety features, is outside the scope of the present specification.

While it is preferred for at least some of the automation apparatus to be separate from the airbag, the automation apparatus may in some embodiments be permanently attached to the airbag. In such embodiments, the automation unit and the airbag may be permanently coupled together where connecting means are described above that require connection, and an end of the retractable means permanently fixed to the airbag in embodiments where the end requires attachment, avoiding need for the operator to secure the automation unit to the airbag.

In embodiments, the cable 26 may be cuttable for the ventilator and a portion of the cable including the fixation barb 38 to be detached from the automation unit. The ventilator and said portion may then be disposed of. A replacement portion may be attached to the automation unit. Alternatively, the automation unit may be separated from the airbag, and in embodiments, other parts of the automation apparatus and reused.

Various connecting means taking different forms (e.g. first and second connecting arrangements) for connecting an actuation unit to an airbag have been described above. Similarly, various fixing means (e.g. first and second fixing pieces) for fixing an end of a retractable means to an airbag or in some embodiments for fixing an actuation unit to a retractable means that is permanently fixed in an airbag, have been described above. In both cases it will be appreciated that the invention is not limited to the particular connecting means and fixing means described.

The airbags described above may be made of rubber or silicone of thickness of between 2 mm and 3 mm. Selection of materials suitable for use in the fixing means and the connecting means in accordance with the embodiments described above will be apparent to the skilled person. In prototypes, many of the parts of the connecting means and the fixing means were made from nylon, although plastic, particularly hard plastic, may be used.

The applicant hereby discloses in isolation each individual feature or step described herein and any combination of two or more such features, to the extent that such features or steps or combinations of features and/or steps are capable of being carried out based on the present specification as a whole in the light of the common general knowledge of a person skilled in the art, irrespective of whether such features or steps or combinations of features and/or steps solve any problems disclosed herein, and without limitation to the scope of the claims.

1. An apparatus for automating repeated collapsing of an airbag for ventilating of a patient using an airbag, the apparatus comprising:
an airbag having an aperture through a first portion thereof;
an elongate retractable member provided for an operator separately from the airbag, for extending in the airbag;
at least one fixing member arranged to enable fixing, by the operator, of an end of the retractable member to a second portion of the airbag through the aperture in the airbag;
an actuation unit, for repeatedly retracting the retractable member at least partially through the aperture in the airbag to pull the second portion towards the first portion, thereby to collapse the airbag, and for enabling or causing expansion of the airbag to an expanded state; and
at least one connector connecting the actuation unit to the airbag, and preventing environment egress of gas from within the airbag between the aperture and the actuation unit.

2. The apparatus of claim 1, further comprising a housing for preventing gas from within the airbag from escaping to the environment via the actuation unit.

3-8. (canceled)

9. The apparatus of claim 1, wherein the at least one fixing member comprises a first fixing member mounted in the airbag at the second portion, and a second fixing member mounted on an end of the retractable member, wherein the first and second fixing members are attachable by the operator.

10. The apparatus of claim 1, wherein the retractable member is sufficiently rigid as to be pushed through the aperture, to push the air bag towards an expanded state, wherein the actuation unit is configured to so push the retractable member, wherein the actuation unit includes a controller and control of the actuation unit to push or retract the retractable member is under the control of the controller.

11. (canceled)

12. The apparatus of claim 1, wherein the retractable member comprises a line, wherein the actuation unit includes means for drawing the line through the aperture and the opening.

13. The apparatus of claim 1, wherein the retractable member comprises a ratchet member and the actuation unit is configured with a gear for mating with the ratchet member to cause movement of the ratchet member.

14-16. (canceled)

17. The apparatus of claim 1, further comprising a controller operatively coupled to the actuation unit, configured to control the actuation unit to draw the retractable member to collapse the airbag wholly or partially, and to permit the airbag to expand wholly or partially.

18. The apparatus of claim 17, further comprising a sensor connected to the controller to provide a signal thereto, wherein the controller is configured to control operation of the actuation unit in dependence on information in the signal, wherein the sensor is locatable and configured to detect at least one of: information indicative of gas pressure within the airbag; information indicative of gas pressure within lungs of a patient; and gas flow rate into the lungs.

19. The apparatus of claim 17, further comprising:
a housing for preventing gas from within the airbag from escaping to the environment via the actuation unit;
a sensor connected to the controller to provide information thereto, wherein the controller is configured to control operation of the actuation unit in dependence on the information, wherein the sensor is a pressure sensor and wherein the sensor is located within the housing to detect pressure within the housing.

20. The apparatus of claim 1, wherein the controller includes circuitry to determine load on the actuation unit, and to control the actuation unit in dependence on the determined load.

21. The apparatus of claim 1, wherein the apparatus is for automating operation of a manual ventilator including the airbag.

22. A method of setting up automated ventilation using an airbag, the apparatus comprising:
fixing, by an operator, using a fixing means, a retractable member provided separately from the airbag to a second portion of the airbag, so as to extend in the airbag through an aperture in a first portion of the airbag;
connecting an actuation unit to the airbag, the actuation unit when connected preventing environmental egress of gas from within the airbag at the aperture;
causing operation of the actuation unit to repeatedly retract the retractable member at least partially through the aperture in the airbag to draw the second portion to the first portion, thereby to collapse the airbag, and to cause or enable expansion of the airbag to an expanded state.

23. An airbag device of a ventilator, comprising:
an airbag;
a first connector mounted in first wall material of an airbag, the first connector defining an aperture therethough and being connectable by an operator to a second connector of an automation unit, such that egress of gas within the airbag is prevented;
a first fixing member mounted in second wall material of the airbag opposite the first connector, wherein the first fixing member is securable by the operator at an interior of the airbag to a second fixing member mounted on the end of a retractable member retractable by the automation unit through the aperture;
a removable closure member sealing the aperture, wherein when so sealed the airbag device can be operated to ventilate manually.

24. The airbag device of claim 23, wherein the first fixing member is securable by the operator to the second fixing member in an action whereby the first fixing member and the second fixing member are pressed together.

25. (canceled)
